(19) United States
(12) Patent Application Publication
(10) Pub. No.: US 2026/0263556 A1
(43) Pub. Date: Sep. 10, 2026

(54) TREG THERAPY

(71) Applicant: Quell Therapeutics Limited, London (GB)

(72) Inventors: Dominik HARTL, Schopfheim (DE); Marc MARTINEZ-LLORDELLA, London (GB)

(21) Appl. No.: 19/165,571

(22) PCT Filed: Mar. 15, 2024
(86) PCT No.: PCT/GB2024/050703

§ 371 Date: Sep. 15, 2025

(30) Foreign Application Priority Data
Mar. 17, 2023 (GB) 2303905.0

(51) Int. Cl. A61K 38/17; A61K 9/51; A61K 38/18; A61K 38/20; A61K 40/11; A61K 48/00; A61P 37/06

(52) U.S. Cl. CPC A61K 38/1709; A61K 9/5123; A61K 38/1808; A61K 38/1841; A61K 38/20; A61K 38/2066; A61K 40/11; A61K 48/0033; A61K 48/005; A61P 37/06

(57) ABSTRACT

The present invention relates to a lipid nanoparticle comprising mRNA encoding FOXP3 and at least a second polypeptide, wherein said lipid nanoparticle comprises a moiety capable of specifically binding to a molecule expressed on a CD4+ T cell and wherein said mRNA is modified to have increased stability as compared to unmodified mRNA.

### FIELD

The present disclosure and invention relate to targeted lipid nanoparticles for in vivo use to deliver mRNA encoding polypeptides/proteins of interest to a target cell. Particularly the invention relates to a targeted lipid nanoparticle comprising mRNA encoding FOXP3 and another protein of interest, typically an antigen or ligand binding receptor. The invention extends to methods of producing the targeted lipid nanoparticles, and to the in vivo use of the targeted lipid nanoparticles.

### BACKGROUND

Adoptive cell therapy (ACT), that is the administration of functional immune cells to a subject, has become an established and evolving immunotherapeutic approach for various medical conditions, including notably malignant or infectious diseases. Tumour-infiltrating lymphocytes were initially shown to be effective in treating metastatic melanoma, and subsequently re-directed T-cells or NK cells expressing chimeric antigen receptors (CARs) or heterologous T-cell receptors (TCRs) to target different cellular target molecules have been developed and adopted for clinical use. Initial approaches used immune cells with cytotoxic properties, e.g. cytotoxic T-cells or NK cells, to target and kill unwanted or deleterious cells in the body, but more recently regulatory T cells (Tregs) have been developed for ACT. Tregs have immunosuppressive function, act to control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases and in transplantation.

The production of Treg immunotherapies typically involves isolation, culture and expansion of Tregs followed by infusion into patients. As part of this process, Tregs may be incubated with cytokines, drugs, other cells or antigens in order to improve their viability and function and/or to confer them with enhanced reactivity against specific antigens. The process often further comprises a step of Treg transduction to introduce exogenous nucleic acid molecules to the cells which may encode an antigen specific receptor, to allow cell targeting. Typically, Tregs may be subjected to genetic engineering to target a predetermined antigen via a chimeric antigen receptor (CAR) or a T cell receptor (TCR).

Tregs numbers within the blood are low (approx. 5-10% of circulating lymphocytes) and since many immunotherapies require doses of several hundreds of millions or even billions of cells, the isolation, culture and expansion of Tregs is a critical part of enabling the production of effective medicaments for patients. In contrast to T effector cell therapies, Treg therapies generally require extensive sorting and selection of cells from PBMCs and extended expansion in culture. Process times of at least 14 days for the production of a therapy are typical, resulting in a high cost of goods. Several different methods of isolation and expansion of Tregs have been reported in the literature. Whilst some methods have only been used in a research and development setting, others have been used under GMP conditions for large scale manufacture. Particularly, isolation under GMP conditions has been carried out using a “CliniMACS” system (CliniMACS TM Instruments, Miltenyi Biotech), which provides a clinical-scale magnetic enrichment of cells in a closed and sterile environment. Reported protocols include carrying out an enrichment step for CD25+ cells, which may be preceded by a depletion step (e.g. of CD8+ cells). However, it has been reported that cell purity may be as low as 80% for such methods, which can be problematic, as the presence of non-Treg cells may be deleterious, especially if the Tregs are to be genetically engineered to have specificity for a target antigen. Other groups have used flow-sorted Tregs in clinical trials, e.g. sorting using CD4+, CD25+ and CD127+. However, flow cytometry methods of isolation are currently carried out using open systems which are difficult to keep sterile and thus are problematic for commercial manufacture. New methods for producing a Treg therapy addressing the issues of ex vivo cell manufacturing are therefore desirable.

The use of mRNA as a therapeutic has been well documented in the literature, where its safety profile and its rapid production have made it an attractive prospect in several therapeutic fields. In 2006, T cells were transduced ex vivo with in vitro transcribed (IVT) mRNA encoding a chimeric antigen receptor specific for CD19, where the cells were shown to be functional in vitro (Rabinovich et al, 2006, Gene Ther., 17, 1027-1035). There are now many examples of T cells which have been ex vivo transduced (electroporated) with mRNA encoding CARs, for example, targeting mesothelin, MCSP, Her-2/neu, ErbB2, folate receptor, CD33, CD123, GD2, EGRF, VEGFR2, CD20, EpCAM, NY-ESO, or MART1. Further, several groups have developed lipid nanoparticle (LNP) technology to improve the stability of mRNA. Veiga et al (2018, Nature Comm., 9, 4493, 1-9) encapsulated mRNA encoding IL10 with LNPs and transduced leukocytes, and Billingsley et al (2020, Nano Lett., 20, 1578-1589) reported the ex vivo generation of CAR T cells using a C14-4 LNP. Additionally, in 2022, Rurik et al (Science, 375, 91-96) produced CAR T cells in vivo using mRNA contained within LNPs targeted to CD5.

However, although there has been much development with regard to use of mRNA to engineer cells for T effector immunotherapy, there has been no report to date of how to utilize this technology in the field of T regulatory cells. Given the small fraction of Treg cells within the blood and the lack of Treg specific markers making the targeting of mRNA/LNPs difficult, it is not surprising that utilization of mRNA for Treg immunotherapy has not yet been documented.

### SUMMARY

The present invention enables the use of mRNA technology for the in vivo production of genetically engineered Tregs, addressing the problems associated with ex vivo production, as discussed above and overcoming the barrier of targeting mRNA in vivo to Tregs. The invention thus allows the production of a cost effective, safe and efficacious Treg therapy which can be used to treat patients with conditions associated with an undesirable inflammatory response, autoimmune disease, or to prevent HvG/GvHD. The present invention overcomes the problems and barriers of the art by specifically targeting mRNA encoding FOXP3 (“FOXP3 mRNA”) to CD4+ T cells in vivo. FOXP3 is a transcription factor which functions as a master regulator in the regulatory pathway in the development and function of regulatory T cells. Transduction of CD4+ T cells with FOXP3 allows conversion of any CD4+ effector cells to cells having a Treg-like suppressive function. By using FOXP3 mRNA in this way, it is only necessary to target CD4+ cells rather than the small Treg cell fraction within the blood. Further, the provision of the FOXP3 mRNA within a LNP decorated with a targeting moiety, allows specific targeting of the CD4+ cells, and their conversion to cells having a Treg-like function. It will be appreciated that any one or more markers specific to CD4+ T cells can be targeted in the present invention using LNP technology, allowing specific delivery of FOXP3 mRNA. The LNP of the invention further comprises mRNA encoding at least one additional protein of interest, particularly an antigen specific receptor allowing the in vivo transduced and converted CD4+ cells to specifically home to a desirable location. The mRNA as utilized in the present invention will typically be modified or comprise modifications to increase its stability and reduce its degradation.

Accordingly, the present invention provides a lipid nanoparticle comprising mRNA encoding FOXP3 and at least a second polypeptide, wherein said lipid nanoparticle comprises a moiety capable of specifically binding to a molecule expressed on a CD4+ T cell and wherein said mRNA is modified to have increased stability as compared to unmodified mRNA.

The lipid nanoparticle (LNP) can be any LNP that is capable of formulation with mRNA and of delivering mRNA to a target cell. Thus, the LNP provides a delivery system for the mRNA, and may protect the mRNA from extracellular degradation whilst allowing cellular uptake and subsequent mRNA release. The LNP is formed from a lipid and includes LNPs based on cationic lipids or ionizable lipids (particularly ionizable lipid derivatives) and LNPs including other types of lipids such as phospholipids, cholesterol and PEG lipids, as discussed in detail further below. The LNP comprises a moiety capable of specifically binding to a molecule which is expressed on a CD4+ T cell. The moiety may be any moiety or molecule capable of binding to a CD4+ cell (e.g., a CD4+ T effector cell or a CD4+ Treg cell), but typically may be an antibody, antibody fragment, ligand for an expressed receptor or an aptamer. The moiety should specifically bind to its target, i.e., should preferably not be capable of binding to other targets or to have reduced specificity for other targets. In a particular embodiment, the moiety is capable of binding CD4, CD62L, CD25, CCR7, CTLA4 and/or CD45RA. The LNP may comprise one or more different moieties, which may bind to any one or more molecules expressed on a CD4+ T cell.

It will be appreciated that the LNP will likely comprise a plurality of moieties (either the same or different types of moieties), which may be capable of binding to one or more molecules expressed by a CD4+ T cell. At least a portion of the plurality of moieties should be present on or at the surface of the LNP to allow binding of the moiety to its CD4+ T cell target. Particularly the CD4+ T cell binding domain of at least one of the plurality of moieties should be present on or at the surface of the LNP. Although possible, it is therefore not necessary for all moieties comprised with the LNP to be present on the surface.

In a particular embodiment, the invention provides a lipid nanoparticle comprising mRNA encoding FOXP3 and at least a second polypeptide, wherein said lipid nanoparticle comprises a moiety capable of specifically binding to CD4 and wherein said mRNA is modified to have increased stability as compared to unmodified mRNA.

As discussed above, the LNP of the invention comprises mRNA encoding FOXP3 and at least a second polypeptide. The FOXP3 mRNA will be translated within the transduced cell to produce FOXP3 protein which will convert any transduced non-Treg CD4+ cell to a cell having a Treg-like phenotype and function. It will be appreciated by a skilled person that converted cells may not be identical to natural Tregs but converted cells have been demonstrated to have a suppressive function. The second polypeptide may be any polypeptide which it may be desirable to express in a Treg therapy. Particularly, the second polypeptide may allow targeting of the transduced cell to a specific location, e.g., tissue within a subject. Thus, the second polypeptide may be an antigen binding receptor, such as a TCR or a CAR. The second polypeptide could alternatively be a further transcription factor which may contribute to conversion of CD4+ effector cells to Treg cells (e.g., Helios), a polypeptide associated with persistence (i.e. which increases persistence of the transduced cell), a cytokine, a polypeptide which is a safety switch, a polypeptide which promotes tissue repair or a polypeptide which promotes suppressive function. It will be appreciated that the LNP of the invention may comprise mRNA encoding more than two polypeptides and thus 3, 4, 5 or 6 different polypeptides may be encoded by the mRNA comprised within the LNP. The mRNA may therefore encode a combination of the polypeptides described above.

In a further embodiment, the present invention therefore provides a lipid nanoparticle comprising mRNA encoding FOXP3 and an antigen binding receptor, particularly a CAR or TCR, wherein said lipid nanoparticle comprises a moiety capable of specifically binding to a molecule expressed on a CD4+ T cell and wherein said mRNA is modified to have increased stability as compared to unmodified mRNA.

Although the LNP provides the mRNA with protection from extracellular degradation, mRNA is generally known to be unstable and may still be susceptible to degradation. The mRNA used in the present invention is therefore modified to improve its stability (extracellular and/or intracellular stability) to provide a robust therapeutic. Any mRNA modifications which achieve the desired effect can be made and include modifications to the 5′ cap and to the 3′ poly A tail. Additional modifications may further be made to the mRNA, for example to enhance translation of the encoded polypeptides.

The LNP of the invention may further comprise other additional molecules, such as miRNA, siRNA or shRNA, which may decrease the endogenous expression of polypeptides adverse to the provision of a suppressive function or Treg phenotype.

Although the LNP of the invention may suitably be used in vivo to provide a suppressive Treg therapy, the invention also encompasses ex vivo uses. Particularly, the invention encompasses ex vivo production of a suppressive cell therapy by combining CD4+ T cells with the LNP described above.

Thus, the present invention provides a composition comprising a CD4+ T cell and a LNP comprising mRNA encoding FOXP3 and at least a second polypeptide, wherein said lipid nanoparticle comprises a moiety capable of specifically binding to a molecule expressed on a CD4+ T cell and wherein said mRNA is modified to have increased stability as compared to unmodified mRNA.

Further, the invention provides an ex vivo method of transducing a CD4+ T cell comprising the step of combining a CD4+ T cell with a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA.

Additionally provided is a transduced CD4+ T cell obtainable or obtained by incubating a CD4+ T cell with a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA.

Alternatively, the invention provides a CD4+ T cell comprising a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA.

The invention further provides cell populations comprising CD4+ T cells of the invention, i.e. CD4+ T cells obtainable by incubating a CD4+ T cell with a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA, and/or CD4+ T cells comprising a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA.

The invention also provides a method of producing a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA, comprising the step of incubating an ionizable or cationic lipid with said mRNA to create a LNP-mRNA formulation. The method may further include a step of conjugating the moiety capable of specifically binding to a molecule expressed on a CD4+ T cell to the LNP, and/or of modifying the LNP and/or moiety so that each comprises a cognate binding partner of a binding partner pair.

As described above, the LNP of the invention, or ex vivo transduced cells or cell populations can be used in vivo to provide a suppressive cell therapy to a subject in need thereof, e.g., a subject having an undesired inflammatory response, a subject suffering from an autoimmune condition or a subject with GvHD or HvG.

Thus, the present invention provides a pharmaceutical composition comprising a LNP, a CD4+ T cell or cell population of the invention.

The pharmaceutical composition may further comprise one or more excipients.

In this respect, the invention provides a method of treating or preventing inflammation, an autoimmune condition or HvG/GvHD in a subject, comprising the step of administering a LNP, a CD4+ T cell, a cell population or a pharmaceutical composition of the invention to the subject. Alternatively, the invention provides a method of promoting tissue remodeling or repair in a subject, comprising the step of administering a LNP, a CD4+ T cell, a cell population or a pharmaceutical composition of the invention to the subject.

The LNP, cell, cell population or pharmaceutical composition of the invention may be administered in one or more doses. It will be appreciated that due to the transient nature of the therapy, treatment of disease conditions requiring long term persistence of suppressive therapy may require the administration of more than one dose, e.g., at least 2, 3 or 4 doses, over a time period.

Alternatively viewed, the invention provides a LNP, a CD4+ cell, a cell population or a pharmaceutical composition of the invention for use in therapy.

More particularly, the invention further provides a LNP, a CD4+ cell, a cell population or a pharmaceutical composition of the invention for use in treating or preventing inflammation, an autoimmune condition or HvG/GvHD or for promoting tissue remodeling or repair in a subject.

In a further aspect, the invention provides use of a LNP, a CD4+ cell, a cell population or a pharmaceutical composition of the invention in the manufacture of a medicament for treating or preventing inflammation, an autoimmune condition or HvG/GvHD or for promoting tissue remodeling or repair in a subject.

### DESCRIPTION OF THE FIGURES

FIG. 1 shows a schematic LNP encompassed by the invention having moieties capable of binding to CD4 and comprising modified mRNA encoding FOXP3 and a CAR.

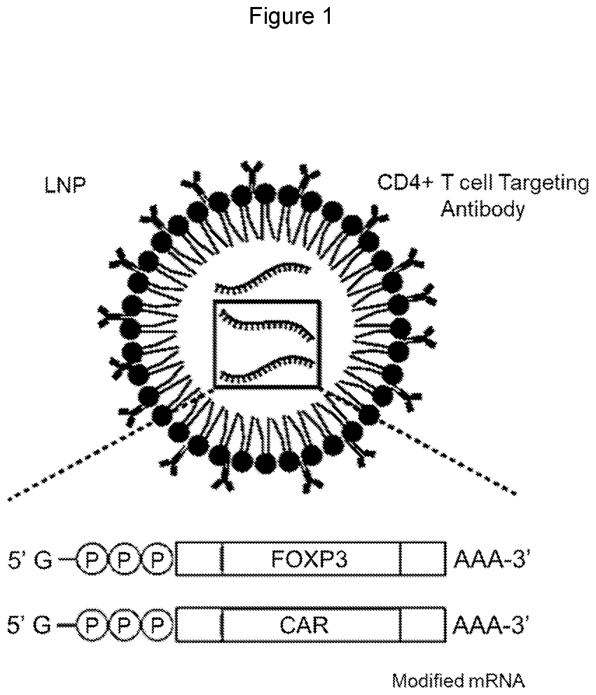

FIG. 2 shows the structure of representative lipids which may be used within a LNP according to the invention (DOTMA, ePC, DLin-MC3-DMA, ALC-0315 and Lipid H) and two additional lipid compounds which may be included in the formulations (Cholesterol and DOPE).

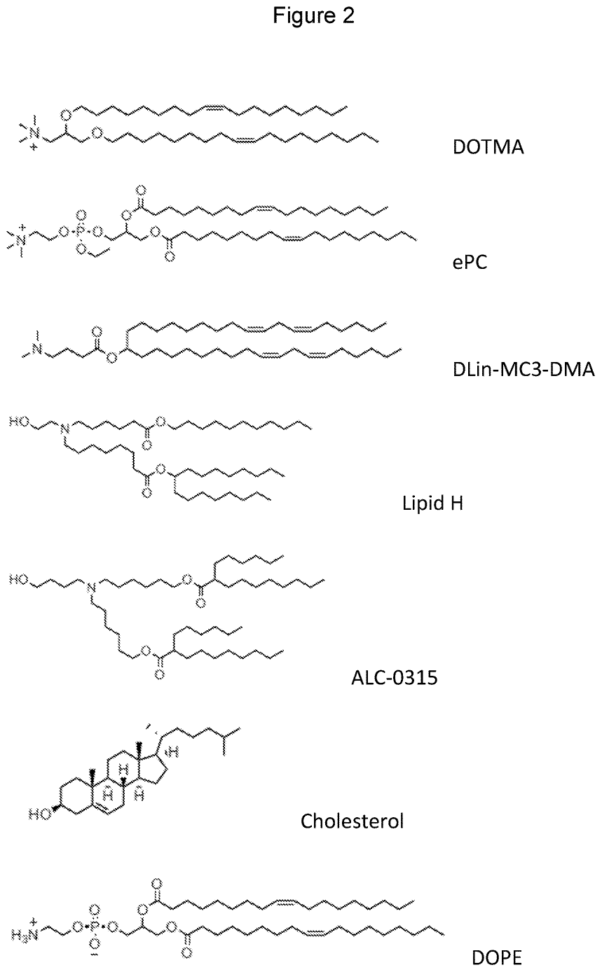

### DETAILED DESCRIPTION

The subject of the products, methods and uses herein is a LNP comprising mRNA encoding FOXP3 and a second polypeptide, which can be used in vivo, but also ex vivo to produce a suppressive cell therapy by transducing CD4+ T cells with the mRNA. The LNP and mRNA modifications protect and provide stability for the mRNA and allow its delivery to specific target cells (CD4+ T cells).

The in vivo utility of the LNP as described herein, in particular, can provide a suppressive cell therapy which is cost effective and does not require ex vivo cell therapy production, addressing many of the issues associated with the manufacture of current Treg or suppressive cell therapies.

A lipid nanoparticle, or LNP, used interchangeably herein refers to a nanoparticle which is formed from one or more lipids, as described below. Reference to a LNP includes lipid-like nanoparticles or nanomaterials (LLNs). Typically, a LNP comprises several different lipids, for example, a cationic or ionizable lipid, together with one or more of a zwitterion lipid, cholesterol, and a PEG-lipid.

A “nanoparticle” refers to particles having a particle size which is less than 1 micrometre. Thus, a nanoparticle may have a particle size (e.g. diameter) of less than 50, 40, 30, 20 or 10 nm. A nanoparticle may have any shape, e.g. spherical, rod, wire, disc, cage, core shell etc. A group or plurality of LNPs of the invention may comprise LNPs having substantially the same or similar shape and/or size or may comprise LNPs having different shapes and/or different sizes. A skilled person will appreciate that the shape/size of the nanoparticle may be dependent on the process used for its manufacture.

A “lipid” is an amphiphilic molecule that contains three domains, namely a polar head group, a hydrophobic tail region and a linker between the two domains. Lipids are generally insoluble in water but soluble in many organic solvents and are usually divided into at least three classes, namely simple lipids including fats and oils, compound lipids, which includes phospholipids and derived lipids, such as steroids. A “lipid” as used herein includes lipid derivatives and lipid-like material which typically comprise more hydrophobic side chains than natural lipids. Typically, a LNP as described herein may be formulated from a cationic or an ionizable lipid, which may be required for encapsulating RNA via electrostatic interactions.

“Cationic lipids” refer to lipids that have a head group with permanent positive charges. Cationic lipids that may used in the LNP of the invention may be quaternary ammonium lipids. Cationic lipids include 1,2-di-O-octadecenyl-3-trimethylammonium-propane (DOTMA) (and analogues thereof such as 1,2-dioleoyl-3-trimethylammonium-propane (DOTAP)); or dimethyldioctadecylammonium bromide (DDAB); 2,3-dioleyloxy-N-[2-(sperminecarboxamido)-ethyl]-N, N-dimethyl-1-propanaminium trifluoro-acetate (DOPSA); ethylphosphatidylcholine (ePC) and 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthrene-3-yl)oxy)carbonyl)amino)-N,N-bis(2-hydroxyethyl)-N-methylethan-1-aminium bromide (BHEM-Cholesterol).

Cationic lipids may be combined with other compounds for use in the LNPs of the invention, for example, with 1,2-dioleoyl-sn-glycero-3-phosphoethanolamine (DOPE), cholesterol and/or carbonate apatite. It will be appreciated that the inclusion of additional compounds may improve or enhance the properties or function of the LNP. For example, inclusion of carbonate apatite may increase the interaction between the LNPs and the cellular membrane.

“Ionizable lipids” refers to lipids which are protonated at low pH, resulting in a positive charge although they remain neutral at physiological pH. Thus, ionizable lipids are generally pH sensitive. Typically, the pH sensitivity allows less interaction with the anionic membranes of blood cells at physiological pH, but the lower pH environment of endosomes promotes a positive charge of the lipids, allowing membrane destabilization and endosomal escape. Examples of ionizable lipids for use in the present invention include (2S)-2,5-bis(3-aminopropylamino)-N-[2-(dioctadecylamino)acetyl]pentanamide (DOGS; Transfectam); N1-[2-((1S)-1-[(3-aminopropyl)amino]-4-[di(3-aminopropyl)amino]butylcarboxamido)ethyl]-3,4-di[oleoyloxy]-benzamide (MV15), DC-Cholesterol, N4-cholesteryl-spermine (GL67); 1,2-dilinoleyloxy-N,N-dimethyl-3-aminopropane (DLin-DMA); 2,2-dilinoleyl-4-dimethylaminoethyl-[1,3]-dioxolane (DLin-KC2-DMA); (6Z,9Z,28Z,31Z)-heptatriaconta-6,9,28,31-tetraen-19-yl4-(dimethylamino)butanoate (DLin-MC3-DMA; MC3). Others that may be used in the present invention include TT3, 5A2-SC8, A18-Iso5-2DC18, LP-01, and cKK-E12. Particular lipid-like derivatives include N¹,N³,N⁵-tris(2-aminoethyl)benzene-1,3,5-tricarboxamide (TT) derivatives, particularly TT3.

Ionizable lipids may be further improved by incorporation of biodegradable lipids, where the biodegradability of lipids may be increased by introducing ester motifs. For example, MC3 may be modified by inserting ester bonds in the linker and lipidic tails resulting in the lipid di((Z)-non-2-en-1-yl)9-((4-(dimethylamino)butanoyl)oxy)heptadecanedioate (L319). Other biodegradable lipids which can be used to produce a LNP of the invention include heptadecane-9-yl8-((2-hydroxyethyl)(8-(nonyloxy)-8-oxyacyl)amino)octanoate (Lipid 5); heptadecane-9-yl8-((2-hydroxyethyl)(6-oxo-6-(undecyloxy)hexyl)amino)octanoate (Lipid H (SM-102)) or ((4-hydroxybutyl)azanediyl)bis(hexane-6,1-diyl)bis(2-hexyldecanoate) (ALC-0315).

Exemplary lipids that can be used in LNPs of the invention are shown in WO2021/077067 (incorporated herein by reference), for example as shown in Formula I-XV.

Zwitterionic ionizable lipids may further be used to form a LNP of the invention, for example, DOPE.

The LNP of the invention may comprise one or more cationic and/or ionizable lipids. Particularly, the LNP may comprise one or more lipids in a concentration range of at least 0.1 mol % to 100 mol %, more particularly at least 10, 20, 30, 40, 50, 60, 70, 80 or 90 mol %.

In addition to cationic or ionizable lipids, the LNPs of the invention may typically comprise other lipid components, for example, phospholipids (such as phosphatidylcholine or phosphatidylethanolamine, DOPE, DSPC, DSPE, SOPC, SOPE etc), neutral or anionic lipids (anionic lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphospatidylethanolamines, lysylphosphatidylglycerols or palmitoyloleyolphosphatidylglycerol), a cholesterol lipid, steroid, steroid analogue, stabilizing lipid or polyethylene glycol (PEG)-functionalized lipids (such as PEG modified phosphatidylethanolamine, PEG modified phosphatidic acid, PEF-modified ceramides, PEG-modified dialkylamines, PEG-modified diacylglycerols or PEG-modified dialkylglycerols, and particularly such as PEG-c-DOMG, PEG-c-DMA or PEG-s-DMG). Particularly the LNP may comprise at least one additional or other lipid component (in addition to the cationic or ionizable lipids). The LNP may therefore comprise 2 or 3 additional lipid components. It will be appreciated by a skilled person, that the inclusion of other lipid components may alter or enhance the functionality or properties of the LNP and a skilled person would be capable of selecting a suitable combination for use in the invention. Particularly, the inclusion of PEG lipids may provide colloidal stability and prevent protein binding to the LNP, the use of neutral or anionic lipids may provide structural stability and define biodistribution, and cholesterol lipids may modulate the bilayer density, fluidity and uptake of the LNP. The additional or other lipids may be present in the LNP in a concentration range of 0.01 mol % to 95 mol %, particularly, at a concentration of at least 10, 20, 30, 40 or 50 mol %.

Lipids or additional lipid components for forming LNPs are commercially available (e.g. at Sigma/Merck) or can be synthesized using known methods, for example, by reacting epoxide-terminated alkyl chains with polyamine cores (available from Avanti Polar Lipids and Enamine, respectively) using Michael addition chemistry. Purification of lipids can be carried out by fractionating lipids using known chromatography systems (e.g., CombiFlash).

The LNP(s) of the invention may be manufactured by several different methods, and many such methods are known and available in the art, for example sonication, stepwise mixing of an ethanolic lipid solution and an aqueous solution of mRNA or microfluidic hydrodynamic focusing. Particularly, LNP(s) may be formed by a method of ethanol dilution as reported in Chen et al, JACS, 2012, 134, 6948-6951 (incorporated herein by reference), by rapidly mixing an alcoholic solution of the lipid and other excipients with an equal volume of aqueous mRNA solution, forming mRNA-LNPs. The mRNA-LNPs may be diluted with aqueous buffer to reduce the ethanol content, where both steps are particularly performed in a microfluidic channel particularly fabricated with polydimethylsiloxane. Microfluidics systems for LNP formation are commercially available, for example, the NanoAssemblr® microfluidic mixing system (Precision Nanosystems Inc). Methods of LNP formation have further been reported in Billingsley et al, JACS, 2020, 20, 1578-1589. LNP(s) of the invention may be further characterized by dynamic light scattering, for example using a Zetasizer Nano ZS, or by use of transmission electron microscopy.

It will be appreciated that the amount of mRNA comprised in an LNP of the invention may vary and is usually dependent on the amount of mRNA used during the formation of the LNP. mRNA content of LNPs can be determine using standard assays, such as using a Quant-iT RiboGreen RNA assay (Invitrogen).

The LNP may comprise one or more moieties capable of specifically binding to a molecule expressed on a CD4+ T cell. Typically, the LNP may comprise a plurality of moieties. It is within the scope of the invention for different moieties to be present within the LNP. Therefore, the LNP may comprise moieties capable of binding to different molecules or different epitopes within a molecule which is expressed by a CD4+ T cell. The LNP of the invention may therefore comprise at least 1, 2, 3, 4, or 5 different moieties.

It will be appreciated that for the LNP to bind to a molecule expressed by a CD4+ T cell, at least one moiety present within the LNP should be present at the surface of the LNP, or at least the binding domain of the moiety (i.e. the portion of the moiety which binds to the molecule expressed by the CD4+ T cell) should be present at the surface of the LNP. Typically, a plurality of moieties (or at least the binding domains of those moieties) will be present at the surface of the LNP to allow binding of the LNP to a CD4+ T cell.

The one or more moieties may bind directly to a molecule expressed by a CD4+ T cell or indirectly, e.g., through one or more other molecules.

The one or more moieties may be conjugated to the LNP of the invention using any known technique. Particularly, the LNP and the moiety may be modified with functioning groups to allow binding of the one or more moieties to the LNP surface. Thus, typically, corresponding binding groups are introduced to the LNP and to the moiety. For example, the LNP may be functionalized to introduce maleimide groups to the surface. Functionalisation may typically occur across the surface of the LNP, although a skilled person will appreciate that functionalization of any part of the surface of the LNP may allow moiety conjugation and thus the production of a target specific LNP. The one or more moieties may be functionalized, for example to introduce sulfhydryl groups (e.g., using N-succinimidyl S-acetylthioacetate (SATA)). Where SATA-maleimide chemistry is used, the one or more moieties may be conjugated to the surface of the LNP using thioester conjugation chemistry.

Alternatively, a domain which binds to the moiety as used in the invention, may be attached to a peptide capable of lipidation for insertion into the LNP. A particular system involves the use of a CDQSSS peptide NIpA motif that undergoes lipidation in bacteria which may be conjugated to any domain capable of binding a moiety of interest, for example to a domain which is capable of binding to the Fc domain of an antibody moiety. An exemplary system is reported in Kedmi et al, Nature Nanotech., 2018, 13, 214-219 (incorporated herein by reference).

The moiety may bind to any molecule expressed by a CD4+ T cell (i.e., any molecule present on the cell surface of a CD4+ T cell). Particularly, the moiety may bind to an endogenously expressed molecule (i.e., a molecule naturally expressed by a CD4+ T cell), e.g., to CD4, CD25, CD62L, CCR7, CTLA4, GITR, CD39, CD73 and/or CD45RA. In a most particular embodiment, the moiety may bind to CD4. In a further particular embodiment, the molecule expressed by a CD4+ T cell may be specifically expressed by a CD4+ T cell (i.e., only expressed by a CD4+ T cell and not expressed by another cell type, or expressed at substantially higher levels by a CD4+ T cell as compared to other cell types, e.g. at least 50, 60, 70, 80 or 90% higher levels of expression). Levels of expression of molecules can be determined using standard methods, such as flow cytometry.

“CD4” as used herein refers to an immunoglobulin co-receptor glycoprotein that assists the TCR in communicating with antigen presenting cells. CD4 is found on the surface of immune cells such as T helper cells and Tregs and includes four immunoglobulin domains (D1 to D4) that are expressed at the cell surface. During antigen presentation, CD4 is recruited, along with the TCR complex, to bind to different regions of the MHC class II molecule (CD4 binds MHC II β2 while the TCR complex binds MHC II αI/βI). Human CD4 typically comprises the sequence of SEQ ID NO. 36.

As discussed above, it is possible for the LNP to bind to more than one molecule expressed by a CD4+ T cell, e.g., CD4 and any one of more of CD25, CD62L, CCR7, CD45RA, CTLA4, GITR, CD73 or CD39, and therefore the LNP in one embodiment may bind to CD4 and CD25, CD4 and CD62L, CD4 and CCR7 or CD4 and CD45RA.

The LNP as used in the present invention comprises a moiety capable of specifically binding to a molecule expressed on a CD4+ T cell. The moiety can be any moiety which is capable of binding to a molecule expressed on a CD4+ T cell. As described previously, the moiety may be selected from the group including but not limited to an antibody, a ligand for a CD4+ T cell receptor or aptamer.

An “antibody” as used herein refers to the various forms of antibody structures including but not being limited to monoclonal and polyclonal antibodies (including full length antibodies), multispecific antibodies (e.g., bi-specific antibodies which may bind to two different molecules expressed on the surface of a CD4+ T cell or to two different epitopes within the same molecule), antibody fragments, immunoadhesins and antibody-immunoadhesin chimeras that specifically recognize (i.e. bind) a target antigen (a molecule expressed on a CD4+ T cell, e.g. a Treg cell). An antibody may be a humanized antibody, chimeric antibody or further genetically engineered antibody, as long as the antibody remains capable of binding to the target antigen. “Antibody fragments” comprise a portion of a full length antibody, particularly the variable domain thereof, or at least the antigen binding site thereof. Examples of antibody fragments include Fab (fragment antigen binding), scFv (single chain fragment variable), single domain antibodies, diabodies, dsFv, Fab′, diabodies, single chain antibody molecules and multispecific antibodies formed from antibody fragments.

“Single chain antibody” (scFv) refers to an engineered antibody consisting of a light chain variable region and a heavy chain variable region connected to one another directly or via a peptide linker sequence. Suitable linkers can be readily selected and can be of any suitable length, such as from 1 amino acid (e.g., Gly) to 30 amino acids, e.g. from any one of 2, 3, 4, 5, 6, 7,8, 9, or 10 amino acids to any one of 12, 15, 18, 20, 21, 25, 30 amino acids, for example, 5-30, 5-25, 6-25, 10-15, 12-25, 15 to 25 etc.

“Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Complementarity determining region” or “CDR” with regard to an immunoglobulin superfamily member, such as an antibody or a TCR, refers to a highly variable loop in the variable region of the heavy chain or the light chain of the immunoglobulin superfamily member. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

The CDRs of the variable regions of a heavy and light chain of an immunoglobulin superfamily member can be predicted from the heavy and light chain variable region sequences of the antibody, using prediction software available in the art, e.g. using the Abysis algorithm, or using the IMGT/V-QUEST software, e.g. the IMGT algorithm (ImMunoGeneTics) which can be found at www.IMGT.org, (see for example Lefranc et al, 2009 NAR 37:D1006-D1012 and Lefranc 2003, Leukemia 17: 260-266). CDR regions identified by either algorithm are considered to be equally suitable for use in the invention. CDRs may vary in length, depending on the immunoglobulin superfamily member from which they are predicted and between the heavy and light chains. Thus, the three heavy chain CDRs of an intact antibody may be of different lengths (or may be of the same length) and the three light chain CDRs of an intact antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 15 amino acids.

It should be noted that the Kabat nomenclature is followed herein where necessary, in order to define the positioning of the CDRs (Kabat et al, 1991, 5th Ed. Public Health Service, National Institutes of Health, Bethesda, MD, 647-669).

Antibodies, and derivatives and fragments thereof, that specifically bind to a target molecule can be prepared using methods well known by those of skill in the art. Such methods include phage display, methods to generate human or humanized antibodies, or methods using transgenic animals or plants engineered to produce human antibodies. Phage display libraries of partially or fully synthetic antibodies are available and can be screened for an antibody or fragment thereof that can bind to a target molecule. Phage display libraries of human antibodies are also available. Once identified, the amino acid sequence or polynucleotide sequence encoding for the antibody (or derivative or fragment thereof) can be isolated and/or determined. The sequence of the antibody can be used to design suitable derivatives or fragments thereof.

The term “humanized antibody” refers to antibodies in which the framework and/or CDRs have been modified to comprise the CDR of an immunoglobulin of different species as compared to that of the parent immunoglobulin. Particularly, a rodent (e.g. mouse) CDR is grafted into the framework region of a human antibody to prepare a humanized antibody. A humanized antibody binds to the same or similar antigen as the donor antibody that provides the CDRs.

Antibodies which bind to molecules expressed on CD4+ T cells, e.g., antibodies which bind to CD4, CD25, CD62L, CTLA4, GITR, CD39, CD73, CCR7 and/or CD45RA are well known in the art and are widely commercially available, such as from Thermo Fisher, Miltenyi Biotech, Invitrogen etc.

Sequences for antibodies which bind to CD4 are also provided in U.S. Pat. No. 8,399,621 (incorporated herein by reference) and include antibodies comprising a VH comprising an amino acid sequence of SEQ ID NO. 1 and a VL comprising an amino acid sequence of SEQ ID NO. 2.

Sequences for antibodies which bind to CD25 are provided in EP3216804 (incorporated herein by reference) and also include an antibody having a heavy chain of SEQ ID NO. 3 and a light chain of SEQ ID NO. 4.

Sequences for antibodies which bind to CCR7 can be found in any of WO2007/00326, WO2012/043533, WO2014/151834 or WO2017/025569 (incorporated herein by reference).

Sequences for antibodies which bind to CD62L can be found in WO1994012215 (incorporated herein by reference), or alternatively, an antibody which binds to CD62L can comprise a heavy chain as set out in SEQ ID NO. 47 and a light chain as set out in SEQ ID NO. 48.

Sequences for antibodies which bind to CTLA4 can be found in WO2017106372, sequences for antibodies which bind to GITR can be found in WO2015184099, sequences for antibodies which bind to CD39 can be found in WO2017157948 and sequences for antibodies which bind to CD73 can be found in WO2016081748 (all of which are incorporated by reference).

A ligand for a CD4+ T cell receptor may include ligands which bind to any receptor expressed by a CD4+ T cell, for example, IL2R. In this regard, the ligand may be a cytokine or a chemokine, or particularly a variant thereof, which specifically binds to a CD4+ T cell. In this embodiment, the ligand may include an IL2 mutein, particularly an IL2 mutein with an increased selective preference for binding to Tregs, as compared to other cell types expressing an IL2R. The IL2 mutein may therefore have increased selectivity to bind to CD25 (IL2α), reduced selectivity to bind to CD122 (IL2Rβ) and/or reduced selectivity to bind to CD132 (IL2Rγ). The IL2 mutein may have at least 10, 20, 30, 40, 50, 60, 70, 80 or 90% increased selectivity to bind to CD25 as compared to unmodified IL2 (e.g. IL2 comprising the sequence of SEQ ID NO. 38). Alternatively or additionally, the IL2 mutein may have at least 10, 20, 30, 40, 50, 60, 70, 80 or 90% reduced selectivity to bind to CD122 and/or CD132 as compared to unmodified IL2 (e.g. IL2 comprising the sequence of SEQ ID NO. 38). Particularly, the IL2 mutein may only comprise epitopes capable of binding to CD25 and may not comprise epitopes capable of binding to CD122 or CD132. A particular IL2 mutein for use in the present invention includes an IL2 mutein having amino acid substitutions of L18R, Q22E and Q126H within SEQ ID NO. 38.

An “aptamer” as used herein refers to a single-stranded oligonucleotide (single-stranded DNA or RNA molecule) that can bind specifically to its target molecule (molecule expressed by a CD4+ T cell). This is typically achieved by intramolecular folding of the single stranded aptamer thereby exhibiting a 3D structure that binds to the target. Aptamers can be readily developed to bind specifically to any target using well known and established methods. Particularly, aptamers can be selected using magnetic bead-based systematic evolution of ligands by exponential enrichment (SELEX) technology, where random DNA libraries containing large numbers of sequences may be screened for binding to a target molecule. Aptamers may be of any length, but typically may be from 20-60 nucleotides, more typically from 30-50, or approximately 40 nucleotides in length.

An exemplary DNA sequence encoding an anti-CD4 aptamer for use in the present invention may comprise or consist of the sequence of SEQ ID NO. 5.

As discussed above, the moiety is capable of specifically binding to a molecule expressed on a CD4+ T cell. “Specific” or “specifically” binding as used herein refers to a moiety which recognizes and binds to a particular target antigen (molecule expressed on a CD4+ T cell) but which does not substantially recognize or bind other antigens, e.g. binds with a greatly reduced affinity compared to the binding to its target molecule, (e.g. with an affinity of at least 10, 50, 100, 500, 1000 or 10000 times less than its affinity for the target molecule). Thus, the moiety as referred to herein may bind to a target molecule expressed on a CD4+ T cell with at least 10, 50, 100, 500, 1000 or 10000 times the affinity of its binding to other proteins. The binding affinity of the moiety can be determined using methods well known in the art such, for example using the Lineweaver-Burk method, or by using commercially available binding model software, such as the 1:1 binding model in the BIAcore 1000 Evaluation software. Suitably, the HBS-P buffer system (0.01M Hepes, pH 7.4, 0.15M NaCl, 0.05% surfactant P20) is used. A moiety which specifically binds to a target antigen from one species, may also bind to that antigen from another species. A moiety that specifically binds to a target antigen may bind also to different allelic forms of the antigen (allelic variants, splice variants, isoforms etc

The LNP as described above, is complexed with mRNA encoding FOXP3 and at least a second polypeptide. Thus, the LNP may encapsulate the mRNA, where the mRNA may be encapsulated in the lipid portion of the LNP or in an aqueous space enveloped by some or all of the lipid portion of the LNP.

“mRNA” or “mRNA polynucleotide” as used interchangeably herein refers to messenger ribonucleic acid which comprises a plurality (i.e., two or more) of linked nucleotides, usually selected from uracil, cytosine, guanine and adenine, or analogues thereof. It will be understood by a skilled person that numerous different mRNA polynucleotides may encode the same polypeptide as a result of the degeneracy of the genetic code. In addition, it is to be understood that the skilled person may, using routine techniques, make nucleotide substitutions that do not affect the polypeptide sequence encoded by the mRNA as defined herein to reflect the codon usage of any particular host organism in which the polypeptide is to be expressed.

An mRNA molecule as used in the present invention may comprise any one or more of a 5′ cap, a 5′ and 3′ UTR, and a poly A tail in combination with the nucleotide sequence/coding sequence encoding the polypeptide of interest e.g., FOXP3 and/or the at least second polypeptide. Further, the mRNA may be circularized.

The 5′ cap is a structure that may be required for mRNA maturation and to allow ribosomal recognition of the mRNA for efficient protein translation. The cap may further provide a stabilization function for the mRNA. Particularly, the mRNA may comprise a 5′ cap1 structure or an analog thereof, which can be produced as discussed below, using Vaccinia capping enzyme and 2′-O-methyltransferase. Commercial kits can be used for introducing a 5′ cap, for example Clean Cap (TriLink, San Diego, CA)

The UTRs are located at the upstream (5′) and downstream (3′) domains of the mRNA coding region and may affect translation efficiency, localization and stability. The UTRs may be between 0-3000 nucleotides in length and may be added to the coding sequence by different methods, including by using primers that anneal to different regions of the UTR. The coding sequence encodes the polypeptide of interest and the poly A tail is usually required for protein translation and mRNA stability. The 5′ and 3′ UTRs may be the naturally occurring endogenous 5′ and 3′ UTRs for the gene of interest or may not be endogenous, and may be incorporated as described above. Modifications to the UTRs may be selected to increase stability of mRNA, for example, UTRs which have reduced amounts of AU can be used. Alternatively, or additionally, modifications may be made to the Kozak sequences found within the 5′ UTR which can affect translation. Thus, modifications can be made which may enhance or increase translation, e.g. by at least 10, 20, 30, 40, 50, 60, 70, 80 or 90% (measured by determining protein levels expressed from the gene of interest). Further, the use of nucleotide analogues in the 5′ or 3′ UTR may increase mRNA stability by inhibiting exonuclease degradation.

The poly A tail may be incorporated into mRNA by including polyTs into the DNA template prior to in vitro transcription (IVT) which is described below. Alternatively, poly A tails can be further extended following IVT by using poly(A) polymerase, such as E. coli poly A polymerase. The poly A tail may comprise at least 5, 10, 20, 30, 50, 100 or 200 nucleotides. The poly A tail may be further modified to increase stability, e.g., by the incorporation of ATP analogs.

As described herein, the mRNA comprised within the LNP is modified mRNA. The mRNA is modified to increase its stability as compared to an equivalent (e.g., the same or a similar) mRNA molecule which has not been modified (unmodified mRNA), particularly when exposed to the same environmental conditions. Particularly, stability may be increased by at least 10, 20, 30, 40, 50, 60, 70, 80 or 90% as compared to an equivalent unmodified mRNA molecule. mRNA stability can be determined by any standard methods known in the art, including by measuring mRNA levels under equivalent conditions using well known techniques, such as Northern blotting. The increase in mRNA stability may refer to the stability of the mRNA whilst complexed or present within the LNP, the intracellular stability of the mRNA or the extracellular ability of the mRNA. Alternatively viewed, the modified mRNA may have an increased half-life or a decreased rate of degradation as compared to an equivalent unmodified mRNA. Particularly, the modified mRNA may have a half-life increased by at least a factor of 2, 3, 4, or 5 as compared to an equivalent unmodified mRNA or may have a rate of degradation at least 10, 20, 30, 40, 50, 60, 70, 80 or 90% less than an equivalent unmodified mRNA.

Any one or more modification(s) can be made to the mRNA that results in the desired effect, i.e. an increase in stability. It will be appreciated that different modifications may be made to the mRNA to provide increased stability, or alternatively viewed to provide protection against degradation. Thus, at least one, two, three, four or five different types of modification may be made to an mRNA molecule as used in the present invention. It will be appreciated, that typically, mRNA is modified prior to being combined or complexed with the LNP.

It will be appreciated, that although the modification(s) made to the mRNA increase its stability, the modification(s) may result in other desirable effects, such as reducing the immunogenicity of mRNA (e.g., by at least 10, 20, 30, 40, 50, 60, 70, 80 or 90%). It is also within the scope of the invention for additional modifications to be made to the mRNA to provide other beneficial properties. Therefore, the mRNA may comprise modifications which do not contribute to an increase in stability but which for example reduce immunogenicity, as long as at least one modification is present which increases stability of the mRNA in accordance with the invention.

Particularly, the mRNA may comprise one or more of the modifications described above, e.g., a 5′ cap or a polyA tail, or may comprise one or more modifications to those structures as already discussed. Additionally, or alternatively, the 5′ and/or 3′ UTRs may be modified as discussed, or the mRNA may be circularised.

The mRNA of the invention may further or alternatively comprise one or more nucleotide modifications. A nucleotide modification refers to mRNA comprising one or more modified nucleotides or nucleosides or nucleobases, particularly chemically modified nucleotides, nucleosides or nucleobases. Nucleotide modifications that can be made to mRNA are well known in the art, e.g., within WO2011/012316 (incorporated by reference herein), where such modifications include the use of chemically modified nucleotides such as pseudouridine, 5-methoxyuridine, 5-methylcytidine, N1-methylpseudouridine, N6-methyladenosine, 2-thiouridine or 5-methyluridine. As described previously a mRNA molecule as used in the invention may comprise any one or more of such chemically modified nucleotides/nucleosides. Typically, chemical modifications are made to uridine and/or to cytidine nucleotides/nucleosides within the mRNA molecule.

Other examples of chemically modified nucleotides/nucleosides that may be used in a mRNA of the invention include thiouridine, N1-methylpseudouridine, 5-hydroxymethylcytidine, 5-hydroxymethyluridine, 5-methylcytidine, 5-methoxyuridine, 5-methoxycytidine, 5-carboxymethulesteruridine, 5-formylcytidine, 5-carboxycytidine, 5-hydroxycytidine, thioguanosine and/or 5-formyluridine.

Particularly, at least 5, 10, 20, 30, 40, 50, 60, 70, 80 or 90% of the uridine nucleotides/nucleoside residues within a mRNA molecule as used herein may be chemically modified. In one embodiment, all of the uridine nucleotides/nucleoside residue within a mRNA molecule as used herein may be chemically modified. Additionally, or alternatively, at least 5, 10, 20, 30, 40, 50, 60, 70, 80 or 90% of the cytosine nucleotides/nucleoside residues within a mRNA molecule as used herein may be chemically modified. In one embodiment, all of the cytosine nucleotides/nucleoside residue within a mRNA molecule as used herein may be chemically modified.

Modifications may be incorporated into a mRNA molecule used within the invention using any well-known method, including by incorporating modified nucleotides during in vitro transcription, e.g. by incorporating any of the modified nucleotides described above, and particularly by incorporating 2-thio-UTP and/or 5-methyl-CTP. Modified nucleotides are commercially available, for example, 2-tho-UTP and 5 methyl-CTP can be obtained from TriLInk Bio Technologies. Different proportions of modified nucleotides may be incorporated into mRNA of the invention.

The mRNA used in the invention can be produced by any method known in the art. For example, mRNA can be produced from a pDNA template that contains a DNA-dependent RNA polymerase promoter and the corresponding sequence for the mRNA construct. pDNA can be amplified within bacterial cells, e.g. E. coli, and purified to provide a pure concentrated, circular pDNA, which is then linearized to serve as a template for the RNA polymerase to transcribe the mRNA. Linearisation may be achieved by mixing the plasmid DNA with a restriction enzyme in a reaction buffer in accordance with well established techniques. Impurities may be removed by tangential flow filtration or by chromatography prior to in vitro transcription using RNA polymerase and nucleotide triphosphates (modified nucleotides can be incorporated as discussed above). The 5′ cap may be added to the produced mRNA either co-transcriptionally or enzymatically. Co-transcriptional capping may be accomplished by adding cap analogs and guanosine triphosphate (GTP) in the transcription mix. Enzymatic capping may be performed after mRNA purification from the in vitro transcription mixture and may use a vaccinia virus capping enzyme to add the capping structure to the mRNA structure. Following in vitro transcription, mRNA may be purified from the impurities using any suitable techniques, such as tangential flow filtration and/or chromatography. Commercial kits are available for mRNA production, such as MEGAScript T7 kit (Invitrogen).

The term “peptide”, “polypeptide” and “protein” are used interchangeably herein and refer to a compound comprised of amino acid residues covalently linked by peptide bonds. A protein or polypeptide should contain at least two amino acids and no limitation is placed on the maximum number of amino acids that can be present.

As discussed above, the mRNA encodes FOXP3 to be expressed in a target cell (a CD4+ T cell) to which the mRNA is delivered. Delivery of mRNA encoding FOXP3 (“FOXP3 mRNA”) as used herein will typically result in an increased level of expression of FOXP3 within a target cell transduced with the mRNA (CD4+ T cell). “FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

“Increasing FOXP3 expression” means to increase the levels of FOXP3 mRNA and/or protein in a cell (or population of cells) in comparison to a corresponding cell which has not been modified (or population of cells). For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Preferably the cell is a CD4+ cell (e.g. a Treg) or the population of cells is a population of CD4+ T cells (e.g. Tregs), as discussed further below in detail. Suitably, the level of FOXP3 mRNA and/or protein in a cell modified by according to the present invention (or a population of such cells) may be increased to at least 1.5-fold greater, 2-fold greater, or 5-fold greater than the level in a corresponding cell which has not been modified according to the present invention (or population of such cells).

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

“FOXP3” or “a FOXP3 polypeptide” is a polypeptide having FOXP3 activity i.e., a polypeptide able to bind FOXP3 target DNA and function as a transcription factor regulating development and function of Tregs. Particularly, a FOXP3 polypeptide may have the same or similar activity to wildtype FOXP3 (SEQ ID NO. 6), e.g., may have at least 40, 50, 60, 70, 80, 90, 95, 100, 110, 120, 130, 140 or 150% of the activity of the wildtype FOXP3 polypeptide. Thus, a FOXP3 polypeptide encoded by the mRNA described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference). As discussed in detail below, FOXP3 or a FOXP3 polypeptide includes functional fragments, variants, and isoforms thereof, e.g., of SEQ ID NO. 6.

A “functional fragment of FOXP3” may refer to a portion or region of a FOXP3 polypeptide or mRNA encoding a FOXP3 polypeptide that has the same or similar activity to the full-length FOXP3 polypeptide or mRNA. The functional fragment may have at least 40%, at least 50%, at least 60%, at least 70%, at least 80%, at least 90%, at least 95%, or 100% of the activity of the full-length FOXP3 polypeptide or mRNA. A person skilled in the art would be able to generate functional fragments based on the known structural and functional features of FOXP3. These are described, for instance, in Song, X., et al., 2012. Cell reports, 1(6), pp. 665-675; Lopes, J. E., et al., 2006. The Journal of Immunology, 177(5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO. 10 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a mRNA encoding a FOXP3 polypeptide, e.g., to SEQ ID NO. 6. FOXP3 variants may have the same or similar activity to a wildtype FOXP3 polypeptide or mRNA, e.g., may have at least 40, 50, 60, 70, 80, 90, 95, 100, 110, 120, 130, 140 or 150% of the activity of a wildtype FOXP3 polypeptide or mRNA. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g., at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO. 6, for example S418E and/or S422A, as described in WO2019/241549 (incorporated herein by reference).

Suitably, the FOXP3 polypeptide encoded by a mRNA molecule as described herein may comprise or consist of the polypeptide sequence of a human FOXP3, such as UniProtKB accession Q9BZS1 (SEQ ID NO: 6), or a functional fragment or variant thereof:

In some embodiments of the invention, the FOXP3 polypeptide comprises or consists of an amino acid sequence which is at least 70% identical to SEQ ID NO: 6 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO: 6 or a functional fragment thereof. In some embodiments, the FOXP3 polypeptide comprises or consists of SEQ ID NO: 6 or a functional fragment thereof.

In some embodiments, as discussed above, the FOXP3 polypeptide may comprise mutations at residues 418 and/or 422 of SEQ ID NO. 6, as follows:

(SEQ ID NO. 7)
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKREQRPSR
CSNPTPGP;
(SEQ ID NO. 8)
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKRSQRPAR
CSNPTPGP;
or
(SEQ ID NO. 9)
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKREQRPAR
CSNPTPGP.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of or a functional variant thereof having at least 80, 85, 90, 95 or 99% identity thereto:

(SEQ ID NO. 10)
GGAHASSSSL NPMPPSQLQL PTLPLVMVAP SGARLGPLPH LQALLQDRPH
FMHQLSTVDA HARTPVLQVH PLESPAMISL TPPTTATGVF SLKARPGLPP GINVASLEWV
SREPALLCTF PNPSAPRKDS TLSAVPQSSY PLLANGVCKW PGCEKVFEEP
EDFLKHCQAD HLLDEKGRAQ CLLQREMVQS LEQQLVLEKE KLSAMQAHLA
GKMALTKASS VASSDKGSCC IVAAGSQGPV VPAWSGPREA PDSLFAVRRH
LWGSHGNSTF PEFLHNMDYF KFHNMRPPFT YATLIRWAIL EAPEKQRTLN
EIYHWFTRMF AFFRNHPATW KNAIRHNLSL HKCFVRVESE KGAVWTVDEL EF

Suitably, the FOXP3 polypeptide may be a variant of SEQ ID NO: 6, for example a natural variant. Suitably, the FOXP3 polypeptide is an isoform of SEQ ID NO: 6. For example, the FOXP3 polypeptide may comprise a deletion of amino acid positions 72-106 relative to SEQ ID NO: 6. Alternatively, the FOXP3 polypeptide may comprise a deletion of amino acid positions 246-272 relative to SEQ ID NO: 6.

Suitably, the FOXP3 polypeptide comprises SEQ ID NO: 11 or a functional variant or fragment thereof.

Suitably the FOXP3 polypeptide comprises or consists of an amino acid sequence which is at least 70% identical to SEQ ID NO: 11 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 11 or a functional fragment thereof. In some embodiments, the FOXP3 polypeptide comprises or consists of SEQ ID NO: 11 or a functional fragment thereof.

Suitably, the FOXP3 polypeptide may be a variant of SEQ ID NO: 11, for example a natural variant. Suitably, the FOXP3 polypeptide is an isoform of SEQ ID NO: 11 or a functional fragment thereof. For example, the FOXP3 polypeptide may comprise a deletion of amino acid positions 72-106 relative to SEQ ID NO: 11. Alternatively, the FOXP3 polypeptide may comprise a deletion of amino acid positions 246-272 relative to SEQ ID NO: 11.

Variants of any amino acid sequence presented herein may have at least 80%, 85%, 90%, 95%, 98% or 99% sequence identity to the reference sequence (i.e., to a reference SEQ ID NO. as specified herein), unless stated otherwise. In particular, such a variant retains the desired or required property of the parent molecule from which it is derived, i.e., the reference sequence. Thus, the variant sequence may have the stated % sequence identity provided that the variant sequence has function.

The term “derivative” or “variant” as used interchangeably herein, in relation to the present proteins or polypeptides includes any substitution of, variation of, modification of, replacement of, deletion of and/or addition of one (or more) amino acid residues from or to the sequence providing that the resultant protein or polypeptide retains the desired function. For example, where the derivative or variant is a transcription factor, the desired function may be the ability of that protein to induce transcription of a particular gene, or where the derivative or variant is an antigen binding receptor, such as a CAR or TCR, the desired function may be activation of the cell in which expression occurs after antigen binding. Alternatively viewed, the variants or derivatives referred to herein are typically functional variants or derivatives. For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding, reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g., increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 1 below.

TABLE 1

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The derivative may be a homologue. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 80%, 85% or 90% identical, preferably at least 95%, 96%, 97%, 98% or 99% identical to the subject sequence. Typically, the variants will comprise the same active sites etc. as the subject amino acid sequence. Although homology can also be considered in terms of similarity (i.e., amino acid residues having similar chemical properties/functions), in the context herein it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence. Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally, e.g., is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Suitably, the FOXP3 mRNA as used in the present invention encodes a FOXP3 polypeptide and may particularly comprise or consist of a polynucleotide sequence set forth in SEQ ID NO: 12:

In some embodiments of the invention, the mRNA polynucleotide sequence encoding the FOXP3 polypeptide or variant may comprise a sequence which is at least 70% identical to SEQ ID NO: 12 or a functional fragment thereof. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 12 or a functional fragment thereof. In some embodiments of the invention, the mRNA encoding the FOXP3 polypeptide or variant may comprise or consist of SEQ ID NO: 12 or a functional fragment thereof.

Suitably, the mRNA encoding a FOXP3 polypeptide may comprise or consist of a polynucleotide sequence set forth in SEQ ID NO: 13:

In some embodiments of the invention, the mRNA polynucleotide encoding the FOXP3 polypeptide or variant comprises a polynucleotide sequence which is at least 70% identical to SEQ ID NO: 13 or a functional fragment thereof. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 13 or a functional fragment thereof. In some embodiments of the invention, the polynucleotide encoding the FOXP3 polypeptide or variant comprises or consists of SEQ ID NO: 13 or a functional fragment thereof.

Suitably, the mRNA polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

The one or more mRNA molecules comprised within the LNP further encode at least one second polypeptide. Thus, the mRNA molecule(s) may encode at least 1, 2, 3, 4, or 5 polypeptides or additional polypeptides.

In a particular embodiment, the mRNA molecule(s) may encode a polypeptide which allows targeting of a cell transduced with the mRNA to a particular location within a subject, e.g. to a particular tissue within a subject. Particularly, the mRNA may encode an antigen binding receptor, such as a CAR or TCR.

“Chimeric antigen receptor” or “CAR” or “CARs” as used herein refers to engineered receptors which confer an antigen specificity onto cells, e.g., CD4+ T cells, particularly Tregs. CARs are also known as artificial T-cell receptors, chimeric T-cell receptors or chimeric immunoreceptors. A CAR typically comprises an extracellular domain comprising an antigen-specific targeting region, termed herein an antigen-binding domain, a transmembrane domain, and an intracellular domain comprising optionally one or more co-stimulatory domains, and an intracellular signaling domain. The antigen-binding domain is typically joined to the transmembrane domain by a hinge domain. The design of CARs, and the various domains that they may contain, is well known in the art.

When the CAR binds its target antigen, this results in the transmission of an activating signal to the cell in which it is expressed. Thus, the CAR directs the specificity of the engineered cells towards the target antigen, particularly towards cells expressing the targeted antigen.

The antigen-binding domain of a CAR may be derived or obtained from any protein or polypeptide which binds (i.e., has affinity for) a desired target antigen, or more generally a desired target molecule. This may be for example, a ligand or receptor, or a physiological binding protein for the target molecule, or a part thereof, or a synthetic or derivative protein. The target molecule may commonly be expressed on the surface of a cell, for example a target cell, or a cell in the vicinity of a target cell (for a bystander effect), but need not be. Depending on the nature and specificity of the antigen binding domain, the CAR may recognise a soluble molecule, for example where the antigen-binding domain is based on, or derived from, a cellular receptor.

The antigen-binding domain is most commonly derived from antibody variable chains (for example it commonly takes the form of a scFv), but may also be generated from T-cell receptor variable domains or, as mentioned above, other molecules, such as receptors for ligands or other binding molecules.

The CAR is typically expressed as a polypeptide also comprising a signal sequence (also known as a leader sequence)), and in particular a signal sequence which targets the CAR to the plasma membrane of the cell. This will generally be positioned next to or close to the antigen-binding domain, generally upstream of the antigen-binding domain. The extracellular domain, or ectodomain, of the CAR may thus comprise a signal sequence and an antigen-binding domain.

The antigen-binding domain provides the CAR with the ability to bind a predetermined antigen of interest. The antigen-binding domain preferably targets an antigen of clinical interest or an antigen at a site of disease.

As noted above, the antigen-binding domain may be any protein or peptide that possesses the ability to specifically recognize and bind to a biological molecule (e.g., a cell surface receptor or a component thereof). The antigen-binding domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/MHC or HLA combination which is capable of binding to the TCRs of Tcon cells active at a site of transplantation, inflammation or disease.

The CAR may be directed towards any desired target antigen or molecule. This may be selected according to the intended therapy, and the condition it is desired to treat. It may for example be an antigen or molecule associated with a particular condition, or an antigen or molecule associated with a cell it is desired to target to treat the condition. Typically, the antigen or molecule is a cell-surface antigen or molecule.

The term “directed against” is synonymous with “specific for” or “anti”. Put another way, the CAR recognises a target molecule. Accordingly, it is meant that the CAR is capable of binding specifically to a specified or given antigen, or target. In particular, the antigen-binding domain of the CAR is capable of binding specifically to the target molecule or antigen (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing the target molecule or antigen, particularly a target cell expressing the target antigen or molecule on its cell surface. Suitably, “specifically binds” as used herein means that the antigen binding domain does not bind to other proteins or binds with a greatly reduced affinity compared to the binding to its target molecule, (e.g. with an affinity of at least 10, 50, 100, 500, 1000 or 10000 times less than its affinity for the target molecule). Thus, the antigen binding domain as referred to herein may bind to a target molecule with at least 10, 50, 100, 500, 1000 or 10000 times the affinity of its binding to other proteins. The binding affinity of the antigen binding domain can be determined using methods well known in the art such, for example using the Lineweaver-Burk method, or by using commercially available binding model software, such as the 1:1 binding model in the BIAcore 1000 Evaluation software. Suitably, the HBS-P buffer system (0.01M Hepes, pH 7.4, 0.15M NaCl, 0.05% surfactant P20) is used.

Antigens which may be targeted by the present CAR include, but are not limited to, antigens expressed on cells associated with transplanted organs, autoimmune diseases, allergic diseases and inflammatory diseases (e.g., neurodegenerative disease). It will be understood by a skilled person that where the cell engineered to express the CAR is a Treg cell, due to the bystander effect of Treg cells, the antigen may be simply present and/or expressed at the site of transplantation, inflammation or disease.

Antigens expressed on cells associated with neurodegenerative disease include those presented on glial cells, e.g., MOG.

Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

In an embodiment the CAR is directed against an HLA antigen, and in particular an HLA-A2 antigen.

Antibodies against such antigens and are known in the art, and conveniently a scFv may be obtained or generated bases on a known or available antibody. In this regard VH and VL, and CDR sequences are publicly available to aid the preparation of such an antibody-binding domain, for example in WO 2020/044055, the disclosure of which is herein incorporated by reference. Any of the antigen binding domains, or CDR, VH, and/or VL sequences disclosed in WO 2020/044055 may be used.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

A representative scFv that could be used in a CAR binding to HLA A2 may comprise or consist of the sequence as set out SEQ ID NO. 14 or a functional variant thereof.

The CAR may comprise a hinge domain, also referred to as the “spacer domain”, which refers to the extracellular part of the CAR that separates the antigen binding domain from the transmembrane domain. The hinge may provide flexibility to access the targeted antigen. For example, long spacers provide extra flexibility to the CAR and allow for better access to membrane-proximal epitopes

Suitable hinge domains will be apparent to those of skill in the art (e.g., Guedan, S., et al., 2018. Molecular Therapy-Methods & Clinical Development, 12, 145-156). Suitable hinge domains include, but are not limited to: CD28 hinge domain, a CD8 hinge domain, an IgG hinge domain, and an IgD hinge domain. Preferably the hinge domain is a CD8 or CD28 hinge domain.

Most preferably, the hinge domain is a CD8 hinge domain. Suitably, the hinge domain may comprise the amino acid sequence shown as SEQ ID NO: 15, or a variant which is at least 80% identical to SEQ ID NO: 15.

Suitably the hinge domain is a CD28 hinge domain. Suitably, the hinge domain may comprise the amino acid sequence shown as SEQ ID NO: 16, or a variant which is at least 80% identical to SEQ ID NO: 16.

The CAR may comprise a transmembrane domain, which refers to the part of the CAR that anchors the CAR into the cell membrane. Thus, the transmembrane domain is capable of spanning or being present within the cell membrane of the cell. The transmembrane domain may be derived from a protein comprising an extracellular and/or intracellular portions and thus the transmembrane domain as used herein may be attached to extracellular and/or intracellular residues derived from the protein of origin, in addition to the portion within or spanning the cell membrane. For example, the transmembrane domain may be attached to a hinge domain derived from the protein of origin e.g., a transmembrane domain derived from CD8 may be attached to a hinge domain derived from CD8. Further, a transmembrane domain derived from CD8 or CD28 for example, may be attached to a CD8 or CD28 costimulatory domain. It will be appreciated by a skilled person that the transmembrane domain can also be synthetic, e.g., de novo designed and not derived from a protein having a transmembrane domain. The presence of a transmembrane domain within a cell membrane can be assessed using any suitable method known in the art, including fluorescence labelling with fluorescence microscopy.

Suitable transmembrane domains will be apparent to those of skill in the art. The transmembrane domain may comprise the transmembrane sequence from any protein which has a transmembrane domain, including any of the type I, type II or type Ill transmembrane proteins. The transmembrane domain of the CAR may also comprise an artificial hydrophobic sequence. The transmembrane domain may be selected so as not to dimerize. Examples of transmembrane (TM) domains used in CAR constructs are: 1) The CD28 TM domain (Pule et al, Mol Ther, 2005, November; 12(5):933-41; Brentjens et al, CCR, 2007, September 15; 13(18 Pt 1):5426-35; Casucci et al, Blood, 2013, Nov. 14; 122(20):3461-72); 2) The OX40 TM domain (Pule et al, Mol Ther, 2005, November; 12(5):933-41); 3) The 41BB TM domain (Brentjens et al, CCR, 2007, Sep. 15; 13(18 Pt 1):5426-35); 4) The CD3 zeta TM domain (Pule et al, Mol Ther, 2005, November; 12(5):933-41; Savoldo B, Blood, 2009, Jun. 18; 113(25):6392-402); 5) The CD8 alpha TM domain (Maher et al, Nat Biotechnol, 2002, January; 20(1):70-5; Imai C, Leukemia, 2004, April; 18(4):676-84; Brentjens et al, CCR, 2007, September 15; 13(18 Pt 1):5426-35; Milone et al, Mol Ther, 2009, August; 17(8):1453-64); 6) the ICOS TM domain; 7) the CD4 TM domain.

Most preferably, the CAR may comprise a CD8 transmembrane domain. Suitably, the transmembrane domain may comprise the amino acid sequence shown as SEQ ID NO: 17, or a variant which is at least 80% identical to SEQ ID NO: 17.

Suitably, the CAR may comprise the CD28 transmembrane domain. Suitably, the transmembrane domain may comprise the amino acid sequence shown as SEQ ID NO: 18, or a variant which is at least 80% identical to SEQ ID NO: 18.

It will be appreciated by a skilled person that a variant of a transmembrane domain, must still be capable of being present across or spanning the cell membrane, i.e., of being a transmembrane domain.

The CAR may comprise an endodomain comprising one or more intracellular signalling domains and optionally one or more co-stimulatory domains.

The “intracellular signalling domain” as used herein refers to the intracellular part of the CAR that participates in transducing the message of the effective CAR binding into the interior of the cell to elicit cell function, e.g., immunosuppressive function.

Suitable intracellular signalling domains will be apparent to those of skill in the art. The intracellular signalling domain is necessary to transduce the effector function signal and direct the Treg to perform its specialized function upon antigen binding. Examples of intracellular signalling domains include, but are not limited to, ζ chain of the T-cell receptor or any of its homologs (e.g., η chain, FcεR1γ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28 or their signalling domains. The intracellular signalling domain may be human CD3 zeta signalling domain, FcγRIII, FcsRI, cytoplasmic tails of Fc receptors, immunoreceptor tyrosine-based activation motif (ITAM) bearing cytoplasmic receptors or combinations thereof.

Most preferably, the intracellular signalling domain may comprise the intracellular signalling domain of human CD3 zeta signalling domain. Suitably, the intracellular signalling domain may comprise the amino acid sequence shown as SEQ ID NO: 19, or a variant which is at least 80% identical to SEQ ID NO: 19.

The CAR may also comprise one or more co-stimulatory domains, which refers to an intracellular part of the CAR that may promote cell function (e.g., immunosuppressive function), expansion, and/or persistence.

Accordingly, the CAR may comprise a compound endodomain comprising a fusion of the one or more co-stimulatory domains to that of an intracellular signalling domain e.g., CD3. Such a compound endodomain may be referred to as a second generation CAR which can transmit an activating and co-stimulatory signal simultaneously after antigen recognition. The co-stimulatory domain most commonly used is that of CD28. This supplies the most potent co-stimulatory signal—namely immunological signal 2, which triggers proliferation. Suitable co-stimulatory domains will be apparent to those of skill in the art.

Accordingly, the CAR preferably comprises a CD28 co-stimulatory domain as set out in SEQ ID NO. 20 or a variant which is at least 80% identical to SEQ ID NO. 20.

Suitably, the one or more co-stimulatory domains may comprise one or more TNF receptor family signalling domain, such as the signalling domain of OX40, 4-1 BB, ICOS or TNFRSF25.

A variant of an intracellular signalling domain and/or of a costimulatory domain may have the same or similar function to the comparative wildtype intracellular signalling domain and/or costimulatory domain, e.g., may have at least 40, 50, 60, 70, 80, 90, 95, 100, 110, 120, 130, 140 or 150% of the function of the wildtype domain (e.g. of the signalling ability of the wildtype domain).

In some embodiments the CAR comprises one or more signal peptides.

The CAR may comprise a leader sequence which targets it to the endoplasmic reticulum pathway for expression on the cell surface. An illustrative leader sequence is a CD8 leader sequence, for example as shown in SEQ ID NO. 21.

A “TCR” or “T Cell Receptor” refers to an immunoglobulin superfamily member comprising a variable binding domain, a constant domain, a transmembrane region and short cytoplasmic tail. The TCR is capable of specifically binding to an antigen peptide bound to a major histocompatibility complex (MHC) encoded receptor. A TCR can be found on the surface of a T cell or within the extracellular milieu in soluble form. A TCR is heterodimeric typically comprised of an alpha-beta or gamma-delta heterodimer, each chain having a constant region and a highly polymorphic variable region comprising three CDRs.

Like other antigen-binding members of the immunoglobulin superfamily, the extracellular portion of TCR chains contain two immunoglobulin domains, a variable domain (e.g. a chain variable domain or Vα, β chain variable domain or Vβ, typically amino acids 1-116 based on Kabat numbering) at the N terminus and one constant domain (e.g. a chain constant domain or Cα, typically amino acids 117-259 based on Kabat, β chain constant domain or Cβ, typically amino acids 117-295 based on Kabat) adjacent to the cell membrane. As discussed above, the variable domains contain CDRs which are separate by framework regions.

The CD4+ cells targeted by the LNPs of the invention may typically express an endogenous TCR. However, the mRNA used in the invention may encode an exogenous TCR, particularly an exogenous TCR comprising alpha and beta chains. The term “exogenous” as used herein refers to a polypeptide or protein which is not naturally expressed by a cell. Typically, the alpha and beta chains of a TCR encoded by mRNA used in the invention may each comprise three CDRs which may be capable of binding to a MHC/HLA and peptide combination expressed at a site of interest, providing a cell expressing the exogenous TCR with a targeting ability. Many exogenous TCRs are known in the art and may be used in accordance with the present invention to provide a transduced cell with a targeting ability. For example, WO2019/202322 (incorporated herein by reference) discloses TCRs which bind to myelin basic protein:HLA complexes, which may be used in the present invention. Autoreactive TCRs isolated from the pancreatic islets of type 1 diabetic organ donors may also be used in the present invention (as reported in Seay et al, JCI insight, 2016, 1(20), e88242), particularly, for example, a TCR specific for GAD65 (555-567) presented in the context of HLA-DR*02:01 may be used, particularly comprising CDR3β amino acid sequence of SEQ ID NO. 39.

The mRNA may further encode a transcription factor which could be relevant to conversion of CD4+ T effector cells to cells having a Treg phenotype. For example, the mRNA may encode one or more of Helios, Sp1, NFAT, AP1, STAT5, CREB, ATF, FOXO1 or Smad.

Particularly, Helios may be encoded by an mRNA molecule as described herein, where Helios may comprise or consist of the amino acid sequence as set out in SEQ ID NO. 22 or a functional variant or fragment thereof (e.g., having at least 80% sequence identity to SEQ ID NO. 22 and having at least 50, 60, 70, 80 or 90% of the transcription factor function of Helios).

The mRNA as used in the present invention may additionally or alternatively encode a polypeptide associated with persistence of the transduced cell, i.e., a polypeptide which increases persistence of the transduced cell, as compared to a cell which does not express the polypeptide associated with persistence (e.g., by at least 10, 20, 30, 40, 50, 60, 70, 80 or 90%). When the polypeptide is expressed, a CD4+ T cell (e.g., Treg) may have increased persistence as compared to a CD4+ T cell (e.g., Treg cell) without the polypeptide. “Persistence” as used herein defines the length of time that cells can survive in a particular environment, e.g., in vivo (e.g. in a human patient or animal model). A CD4+ T cell as disclosed herein may have at least 10, 20, 30, 40, 50, 60, 70, 80 or 90% increased persistence as compared to a CD4+ which does not express the polypeptide. Persistence can be measured by for example, determining the amount or numbers of transduced cells within a subject or patient over time, where cells expressing the polypeptide are compared to equivalent cell types which do not express the polypeptide, or compared to non-engineered cells. It is possible to track transduced cells, for example, using a marker protein, e.g. CD34 for cells which also express a RQR8 safety switch.

Persistence in T cells may also be determined by measuring the level of pSTAT5 signaling within a cell or a cell population. An increase in pSTAT5 signaling is associated with an increase in persistence, e.g., an increase in pSTAT5 signaling of at least 10, 20, 30, 40, 50, 60, 70, 80 or 90%. pSTAT5 signaling can be measured using standard assays.

STAT5 is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation to the IL2R results in STAT5 phosphorylation by activating the Jak1/Jak2 and Jak3 kinases via specific signalling domains present in the IL-2Rβ and IL-2Rγ chain, respectively. Although JAK1 (or JAK2) can phosphorylate STAT5 without the need of JAK3, STAT5 activity is increased by the transphosphorylation of both JAK1/JAK2 and JAK3, which stabilizes their activity. Phosphorylated STAT5 is capable of homodimerization, resulting in a functional transcription factor.

Several technologies have been reported that may have utility for increasing persistence within CD4+ T cells and particularly within Treg cells. For example, technologies have been reported based on providing a constitutive STAT5 signal to a transduced cell. Such technologies include the use of modified JAK and/or STAT5 molecules, which may for example be constitutively phosphorylated or dimerized. WO2017/218850 (incorporated herein by reference) discloses a modified STAT5b molecule, STAT5bCA which allows constitutive STAT5 signalling within Treg cells in which it is expressed. Further, WO2019/053420 (incorporated herein by reference) discloses the use of STAT5 molecules which are engineered to comprise a dimerization domain, allowing spontaneous dimerization between molecules, (e.g., the STAT5 molecules may be engineered to comprise a leucine zipper), or alternatively may be engineered to comprise a gain of function mutation, such as a S710F mutation in STAT5.

Other persistence technologies that have been reported are based on chimeric polypeptides comprising a STAT5 association motif and a JAK1 and/or JAK2 binding motif.

“STAT5 association motif” as used herein refers to an amino acid motif which comprises a tyrosine and, upon phosphorylation of the tyrosine, is capable of binding a STAT5 polypeptide. The STAT5 association motif may exist endogenously in a cytoplasmic domain of a transmembrane protein, for example a cytokine receptor, particularly IL2R. Particularly the endodomain of the IL2Rβ chain (or a truncated portion thereof) comprises a STAT5 association motif and a JAK1 binding motif and can be used in persistence technologies to provide pSTAT5 signalling.

“JAK1-binding motif” as used herein refers to a BOX motif which allows for tyrosine kinase JAK1 association. Analogously, “JAK2binding motif” as used herein refers to a BOX motif which allows for tyrosine kinase JAK2 association. Suitable JAK1- and JAK2-binding motifs are described, for example, by Ferrao & Lupardus (Frontiers in Endocrinology; 2017; 8(71); which is incorporated herein by reference).

WO2020/044055 (incorporated herein by reference) relates to a chimeric antigen receptor comprising a STAT5 association motif and a JAK1 binding motif within the endodomain of the CAR. Particularly, the endodomain of the CAR comprises the endodomain or a truncated endodomain from the IL2RB polypeptide. Other technologies that may be encoded by an mRNA for use in the invention, include chimeric cytokine receptors, including for example a STAT5 association motif and JAK1 and/or JAK2 binding motif within the endodomain and an exodomain which may be from a different protein to the endodomain (e.g. the exodomain may comprise the exodomain of a different cytokine receptor from cytokine receptor from which the endodomain is derived). Examples of chimeric cytokine receptors that could be used in the invention are described in WO2018/038945, WO2017/029512, and WO2012/138858 (incorporated herein by reference). Persistence technologies based on the use of exogenous small molecules for induction of signal have also been reported and could be used in the present invention, such as those disclosed in WO2019/169290 (incorporated herein by reference).

One or more cytokines may be encoded by the mRNA used in the present invention. A “cytokine” as referred to herein is a cell signalling molecule that regulates the immune system's response to inflammation and infection and aids cell to cell communication in immune responses. Examples of cytokines include chemokines, interferons, interleukins, lymphokines and tumour necrosis factors. Particular cytokines which may be encoded include any one or more of IL10, IL35, IL33, TGFβ, IL37 and IL22.

The mRNA may therefore encode IL10 comprising or consisting of the sequence as set out in SEQ ID NO. 23 or a functional variant or fragment thereof (e.g., with at least 80% identity to SEQ ID NO. 23).

The mRNA may further or alternatively encode IL35 (IL12alpha) comprising or consisting of the sequence as set out in SEQ ID NO. 24 or a functional variant or fragment thereof (e.g. with at least 80% identity to SEQ ID NO. 24).

The mRNA may encode TGFbeta comprising or consisting of the sequence as set out in SEQ ID NO. 25 or a functional variant or fragment thereof (e.g., with at least 80% identity to SEQ ID NO. 25).

The mRNA may encode IL33 comprising or consisting of the sequence as set out in SEQ ID NO. 40 or a functional variant or fragment thereof (e.g., with at least 80% identity to SEQ ID NO. 40).

The mRNA may encode IL37 comprising or consisting of the sequence as set out in SEQ ID NO. 49 or a functional variant or fragment thereof (e.g., with at least 80% identity to SEQ ID NO. 49).

The mRNA may encode IL22 comprising or consisting of the sequence as set out in SEQ ID NO. 50 or a functional variant or fragment thereof (e.g., with at least 80% identity to SEQ ID NO. 50).

The mRNA may encode a safety switch. The safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, which when expressed allows the cell to be deleted to turn off expression of the transgene. A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9. However, it is preferred for the suicide moiety to be, or to comprise, an epitope which is recognised by a cell-deleting antibody or other binding molecule capable of eliciting deletion of the cell. In such an embodiment, the safety switch polypeptide is expressed on the surface of a cell.

The term “delete” as used herein in the context of cell deletion is synonymous with “remove” or “ablate” or “eliminate” The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. Biosimilars for Rituximab are available and may be used. A person of skill in the art is readily able to use routine methods to prepare an antibody having the binding specificity of Rituximab using the available amino acid sequences therefor.

Cells, which also express a safety switch polypeptide comprising this sequence can be selectively killed using the antibody Rituximab, or an antibody having the binding specificity of Rituximab. The safety switch polypeptide is expressed on the cell surface and when the expressed polypeptide is exposed to or contacted with Rituximab, or an antibody with the same binding specificity, death of the cell ensues.

For example, the suicide constructs of WO2013/153391 or PCT/EP2021/064053 (both incorporated herein by reference) may be used in a cell or cell population (e.g., a CD4+ T cell, Treg or Treg population) as described herein.

The mRNA used in the present invention may encode one or more polypeptides capable of promoting tissue repair. Such polypeptides include amphiregulin (areg), CCN2 or CCN3 or functional variants or fragments thereof. The mRNA may encode Areg comprising or consisting of sequence as set forth in SEQ ID NO. 26 or a functional variant or fragment thereof (e.g., with at least 80% identity to SEQ ID NO. 26).

The mRNA may encode CCN2 comprising or consisting of sequence as set forth in SEQ ID NO. 41 or a functional variant or fragment thereof (e.g., with at least 80% identity to SEQ ID NO. 41).

The mRNA may encode CCN3 comprising or consisting of sequence as set forth in SEQ ID NO. 27 or a functional variant or fragment thereof (e.g., with at least 80% identity to SEQ ID NO. 27).

The mRNA as used in the invention may encode one more polypeptides capable of increasing suppressive function of a cell expressing the polypeptide. Such polypeptides include CTLA4, LAG3, PDL1/PDL2, ICOS, CD73 or CD39.

The mRNA may therefore encode CTLA4 comprising or consisting of the sequence as set forth in SEQ ID NO. 42 or a functional variant or fragment thereof (e.g., with at least 80% identity to SEQ ID NO. 42).

The mRNA may therefore encode LAG3 comprising or consisting of the sequence as set forth in SEQ ID NO. 43 or a functional variant or fragment thereof (e.g., with at least 80% identity to SEQ ID NO. 43).

The mRNA may therefore encode PDL1 comprising or consisting of the sequence as set forth in SEQ ID NO. 44 or a functional variant or fragment thereof (e.g., with at least 80% identity to SEQ ID NO. 44).

The mRNA may therefore encode PDL2 comprising or consisting of the sequence as set forth in SEQ ID NO. 45 or a functional variant or fragment thereof (e.g., with at least 80% identity to SEQ ID NO. 45).

The mRNA may therefore encode ICOS comprising or consisting of the sequence as set forth in SEQ ID NO. 46 or a functional variant or fragment thereof (e.g., with at least 80% identity to SEQ ID NO. 46).

The mRNA may therefore encode CD73 comprising or consisting of the sequence as set forth in SEQ ID NO. 28 or a functional variant or fragment thereof (e.g., with at least 80% identity to SEQ ID NO. 28).

Alternatively, or additionally the mRNA may encode CD39 comprising or consisting of the sequence as set forth in SEQ ID NO. 29 or a functional variant or fragment thereof (e.g., with at least 80% identity to SEQ ID NO. 29).

The LNP of the invention comprises mRNA which encodes FOXP3 and at least a second polypeptide. The FOXP3 and at least the second polypeptide may be encoded by a single mRNA molecule, or by separate mRNA molecules comprises within the LNP. Thus, in one embodiment, the LNP may comprise a single mRNA molecule encoding FOXP3 and at least a second polypeptide (e.g., an antigen binding receptor such as a CAR or TCR), or may comprise two or more distinct mRNA molecules, each encoding a separate polypeptide (e.g., a LNP may comprise one mRNA molecule encoding FOXP3 and a second mRNA molecule encoding an antigen binding receptor, e.g., a CAR or TCR). Where further polypeptides are encoded, additional separate mRNA molecules may be present encoding such polypeptides (e.g., encoding a persistence associated polypeptide, a cytokine, a safety switch polypeptide etc), or such polypeptides may be encoded by a single mRNA molecule which also encodes FOXP3 and the second polypeptide. Alternatively, the mRNA molecules encoding FOXP3 and the second polypeptide may be provided separately, but such mRNA molecules may further encode, third, fourth or other polypeptides as desired. Where separate mRNA molecules are complexed with the LNP, it is possible to provide these in different ratios or amounts, if different expression levels of polypeptides are desired.

Where two or more polypeptide sequences are expressed from a single mRNA molecule, they may be linked by a sequence allowing co-expression of the two or more coding sequences. In particular, the co-expression sequence, or alternatively termed, the co-expression site, may enable expression of an encoded protein or polypeptide as a discrete entity.

In particular the co-expression sequence may encode a self-cleavage sequence in between encoded polypeptides. Particularly, the self-cleaving sequence may be a self-cleaving peptide. Such sequences auto-cleave during protein production. Self-cleaving peptides which may be used are 2A peptides or 2A-like peptides which are known and described in the art, for example in Donnelly et al., Journal of General Virology, 2001, 82, 1027-1041, herein incorporated by reference. 2A and 2A-like peptides are believed to cause ribosome skipping, and result in a form of cleavage in which a ribosome skips the formation of peptide bond between the end of a 2A peptide and the downstream amino acid sequence. The “cleavage” occurs between the Glycine and Proline residues at the C-terminus of the 2A peptide meaning the upstream cistron will have a few additional residues added to the end, while the downstream cistron will start with the Proline.

Suitable self-cleaving domains include P2A, T2A, E2A, and F2A sequences as shown in SEQ ID Nos: 30-33 respectively. The sequences may be modified to include the amino acids GSG at the N-terminus of the 2A peptides. Thus, also included as possible options are sequences corresponding to SEQ ID Nos. 30-33, but with GSG at the N termini thereof. Such modified alternative 2A sequences are known and reported in the art. Alternative 2A-like sequences which may be used are shown in Donnelly et al (supra), for example a TaV sequence.

The self-cleaving sequences included in the mRNA molecule(s) may be the same or different.

The self-cleaving sequence may include an additional cleavage site, which may be cleaved by common enzymes present in the cell. This may assist in achieving complete removal of the 2A sequences after translation. Such an additional cleavage site may for example comprise a Furin cleavage site. Such cleavage sites are known in the art, and may include for example RXXR (SEQ ID NO: 34), for example RRKR (SEQ ID NO: 35).

The nucleic acid molecule/polynucleotides used herein may be codon-optimised. Codon optimisation has previously been described in WO 1999/41397 and WO 2001/79518 (both incorporated herein by reference). Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

The LNP of the invention may further comprise additional molecules or components, for example, miRNA, siRNA, or shRNA. Particularly, whilst the mRNA comprised with the LNP may increase the expression of particular polypeptides within a CD4+ T cell, a skilled person will appreciate that it may be desirable to additionally reduce or decrease the expression of particular endogenous polypeptides/proteins, particularly those which may be adverse to the provision of a suppressive function or a Treg phenotype. This can be achieved using molecules such as miRNA, or siRNA which are well understood in the art. Typically, miRNA molecules for example are relatively short (on average 22 nucleotides) and may interact with the 3′ UTR of target mRNA to suppress translation. siRNAs are again short RNA molecules (usually 20-24 nucleotides long), but which are complementary to a target mRNA molecule and induce mRNA cleavage. miRNAs or siRNAs which reduce protein/mRNA levels (e.g., reduction by at least 10, 20, 30, 40, 50, 60, 70, 80 or 90% as compared to the same without the introduced miRNA or siRNA) of Tbet, RORγ and/or Gata3 may be included within an LNP of the invention.

Although a primary focus of the invention is to use the LNPs of the invention in vivo to generate a suppressive cell therapy, obviating the need to carry out ex vivo manufacture, it is possible to use the LNPs to produce ex vivo transduced cells which may be useful as a suppressive therapy.

Thus, in this respect, the invention provides an ex vivo method of transducing a CD4+ T cell comprising the step of combining a CD4+ T cell with a LNP comprising mRNA encoding FOXP3 and at least a second polypeptide, wherein said LNP comprises a moiety capable of specifically binding to a molecule expressed on a CD4+ T cell and wherein said mRNA is modified to have increased stability as compared to unmodified mRNA.

It will be appreciated by a skilled person that the step of combining a CD4+ T cell with a LNP of the invention is of sufficient length to allow transduction of the CD4+ T cell with the mRNA comprised within the LNP, e.g., at least 1 hour, 2 hours, 5 hours, 24 hours, or 48 hours.

Cells may be activated and/or expanded prior to, or after, the introduction of LNP/mRNA as described herein, for example by treatment with an anti-CD3 monoclonal antibody or both anti-CD3 and anti-CD28 monoclonal antibodies. The cells may also be expanded in the presence of anti-CD3 and anti-CD28 monoclonal antibodies in combination with IL-2. Suitably, IL-2 may be substituted with IL-15. Other components which may be used in a cell (e.g. Treg) expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

A “CD4+ T cell” as used herein is a type of lymphocyte having an endogenously expressed TCR on the cell surface. CD4+ T cells additionally express CD4 on the cell surface. CD4+ T cells include T helper cells which become activated when presented with peptide antigens by MHC class II molecules on the surface of antigen presenting cells (APCs). These cells may differentiate into one of several subtypes including Th1, Th2, h3, Th17, Th9 or TFH which secrete different cytokines to facilitate different types of immune responses. CD4+ T cells may include memory T cells that represent a subset of antigen specific T cells that persist long-term after an immune response has been resolved. They quickly expand to large numbers upon re-exposure to their cognate antigen. Memory T cells comprise three subsets, central memory T cells (TCM cells), TEM cells and TEMRA cells. Memory cells typically express the marker CD45RO.

A CD4+ T cell as used herein may further be a Treg.

Several different subpopulations of Tregs have been identified which may express different or different levels of particular markers. Tregs generally are T cells which express the markers CD4, CD25 and FOXP3 (CD4⁺CD25⁺FOXP3⁺).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3+ T cell, a CD4⁺CD25⁺CD127⁻ T cell, or a CD4⁺CD25⁺FOXP3⁺CD127^{-/low} T cell.

Suitably, the Treg may be a natural Treg (nTreg). As used herein, the term “natural T reg” means a thymus-derived Treg. Natural T regs are CD4⁺CD25⁺FOXP3⁺Helios⁺Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Further suitable Tregs include, but are not limited to, Tr cells (which do not express Foxp3, and have high IL-10 production).

Different subpopulations of Tregs are known to exist, including naïve Tregs (CD45RA⁺FoxP3^{low}), effector/memory Tregs (CD45RA⁻FoxP3^{high}) and cytokine-producing Tregs (CD45RA⁻FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g., at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g., less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc., expression may refer to cell surface expression, i.e., the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g., a memory Treg cell). Alternatively viewed, a naïve Treg cell may express at least 2, 3, 4, 5, 10, 50 or 100-fold the amount of CD45RA as compared to a non-naïve Treg cell (e.g., a memory Treg cell). The level of expression of CD45RA can be readily determined by methods of the art, e.g., by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor.

Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g., at least 10, 20, 30, 40, 50, 60, 70, 80 or 90% lower level of expression.

Naïve Tregs may further express additional markers, including CCR7⁺ and CD31⁺

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out, and methods for determining the presence and/or levels of expression of cell markers are well-known in the art and include, for example, flow cytometry, using commercially available antibodies.

Suitably, a CD4+ T cell, such as a Treg, for use in an ex vivo method of the invention, is isolated from peripheral blood mononuclear cells (PBMCs) obtained from a subject. Suitably the subject from whom the PBMCs are obtained is a mammal, preferably a human. If the cell is for administration to a subject after transduction, then the cell may be matched (e.g., HLA matched) or may be autologous to the subject to whom the transduced cell is to be administered. Suitably, the subject to be treated is a mammal, particularly a human. The cell may be generated ex vivo either from a patient's own peripheral blood (1^{st} party), or in the setting of a haematopoietic stem cell transplant from donor peripheral blood (2^{nd} party), or peripheral blood from an unconnected donor (3^{rd} party).

Suitably, the CD4+ T cells (e.g., Treg) is part of a population of cells. It will be appreciated that although the cell population comprises the CD4+ T cells as described herein, other cell types may also be present within the population and therefore the population may not be a pure population of CD4+ T cells. However, in a particular embodiment the population may comprise at least 30, 40, 50, 60, 70, 80 or 90% CD4+ T cells. Suitably, the population of cells comprising CD4+ T cells, comprises at least 70% Tregs, such as at least 75, 85, 90, 95, 97, 98 or 99% Tregs. Such a population may be referred to as an “enriched Treg population”.

In some aspects of the ex vivo methods of the invention, the CD4+ T cell (e.g., T reg) may be derived from ex-vivo differentiation of inducible progenitor cells (e.g., iPSCs) or embryonic progenitor cells to the Treg. Suitable methods for differentiation are known in the art and include that disclosed in Haque et al, J Vis Exp., 2016, 117, 54720 (incorporated herein by reference).

The targeted CD4+ T cell may thus either be a non-Treg CD4+ cell or a Treg cell as defined above. A non-Treg CD4+ T cell generally does not have suppressive activity and is considered to be a CD4+ T conv cell as defined below. Thus, either type or both types of cells may be transduced in vivo or ex vivo in accordance with the present invention. The invention encompasses the transduction of a population of CD4+ cells, where such a population may comprise both non-Treg CD4+ T cells and Treg cells, or may comprise only non-Treg CD4+ T cells or only Treg cells. Populations may for example comprise at least 5, 10, 20, 30, 40, 50, 60 or 70% Treg cells. As discussed herein, transduction of non-Treg CD4+ T cells may result in conversion of those cells from having a conventional T cell function to having a suppressive function by the expression of FOXP3. Such converted cells having a suppressive function are referred to herein as T-reg like cells. Transduced cells of the invention having a suppressive function may provide a suppressive cell therapy.

The ex vivo method of transducing a CD4+ T cell with a LNP of the invention may result in the conversion of non-Treg CD4+ T cells to cells having a suppressive function. It will be appreciated by a skilled person that not all cells incubated with the LNP may be transduced and that it is possible that only a portion of cells will be transduced (e.g. at least 30, 40 or 50%). It will further be appreciated that not all transduced non-Treg cells may be converted to have a suppressive function. However, preferably, at least 70, 80, 90 or 95% of transduced non-Treg CD4+ cells may be converted to have a suppressive function. Transduced Treg cells may have an enhanced or increased suppressive function as compared to Treg cells which are not transduced (e.g., an increase in suppressive function of at least 10, 20, 30, 40, 50, 60, 70, 80 or 90%).

“Suppressive”, “suppressive function” or “immunosuppressive function” as used herein may refer to the ability of a cell (e.g., a Treg cell, or a Treg like cell (e.g., a CD4+ T cell expressing FOXP3)) to reduce or inhibit one or more of a number of physiological and cellular effects facilitated by the immune system in response to a stimulus such as a pathogen, an alloantigen, or an autoantigen. Examples of such effects include increased proliferation of conventional T cell (Tcon) and secretion of proinflammatory cytokines. Any such effects may be used as indicators of the strength of an immune response. A relatively weaker immune response by Tconv in the presence of a cell (e.g., a Treg cell, or a Treg like cell (e.g., a CD4+ T cell expressing FOXP3)) would indicate an ability of that cell to suppress immune responses. For example, a relative decrease in cytokine secretion would be indicative of a weaker immune response, and thus indicative of the ability of a cell (e.g., a Treg cell, or a Treg like cell (e.g., a CD4+ T cell expressing FOXP3)) to suppress immune responses. Tregs in particular can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of a cell (e.g., a Treg cell, or a Treg like cell (e.g., a CD4+ T cell expressing FOXP3)). In particular, antigen-specific Tconv cells may be co-cultured with cells, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured cells.

As used herein, the term “conventional T cell” or Tcon or Tconv (used interchangeably herein) means a T lymphocyte cell which expresses an as T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues.

Antigen-specific Tconv cells co-cultured with transduced cells as referred to herein may proliferate 5%, 10%, 20%, 30%, 40%, 50%, 60%, 70%, 90%, 95% or 99% less than the same Tconv cells cultured in the absence of the transduced cells. For example, antigen-specific Tconv cells co-cultured with the present transduced cells may proliferate 5%, 10%, 20%, 30%, 40%, 50%, 60%, 70%, 90%, 95% or 99% less than the same Tconv cells cultured in the presence of non-engineered cells (e.g. CD4+ non-engineered cells). The cells comprising the mRNA as defined herein, e.g., Tregs, may have an increased suppressive activity as compared to non-engineered cells, e.g., Tregs (e.g. an increased suppressive activity of at least 5, 10, 20, 30, 40, 50, 60, 70, 80 or 90%).

Antigen-specific Tconv cells co-cultured with the transduced cells herein may express at least 10%, at least 20%, at least 30%, at least 40%, at least 50%, or at least 60% less effector cytokine than corresponding Tconv cells cultured in the absence of the transduced cells (e.g. in the presence of non-engineered cells). The effector cytokine may be selected from IL-2, IL-17, TNFα, GM-CSF, IFN-γ, IL-4, IL-5, IL-9, IL-10 and IL-13. Suitably, the effector cytokine may be selected from IL-2, IL-17, TNFα, GM-CSF and IFN-γ.

The invention further encompasses a pharmaceutical composition comprising (i) the LNPs of the invention, (ii) CD4+ T cells transduced ex vivo with LNPs of the invention obtainable as described above or a cell population comprising the cells, or (iii) CD4+ T cells comprising LNPs of the invention or a cell population comprising the cells.

Pharmaceutical compositions may be prepared by any method known in the art, including combining the active ingredient (i.e., the LNPs or transduced cells) with a carrier or any desirable excipient. Pharmaceutical compositions according to the invention may be developed that are suitable for administration to any animal subject, including humans and other primates, domestic animals, such, cats and dogs and farm animals such as sheep, pigs and cows. The LNPs may further be for administration to mice, rats and rabbits.

A pharmaceutical composition is a composition that comprises or consists of a therapeutically effective amount of a pharmaceutically active agent i.e., the LNP, cell (e.g. Treg or suppressive cell), or cell population. It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The LNP, cells, cell population or pharmaceutical compositions may be administered in a manner appropriate for treating and/or preventing the desired disease or condition. The quantity and frequency of administration will be determined by such factors as the condition of the subject, and the type and severity of the subject's disease or condition, although appropriate dosages may be determined by clinical trials. The pharmaceutical composition may be formulated accordingly.

The LNPs, cell, cell population or pharmaceutical composition as described herein can be administered parenterally, for example, intravenously, or they may be administered by infusion techniques. The LNP, cell, cell population or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise LNPs or cells in infusion media, for example sterile isotonic solution. The pharmaceutical composition may be enclosed in ampoules, disposable syringes or multiple dose vials made of glass or plastic.

The LNP, cell, cell population or pharmaceutical composition may be administered in a single or in multiple doses. Particularly, the LNP, cell, cell population or pharmaceutical composition may be administered in a single, one off dose. The pharmaceutical composition may be formulated accordingly.

The pharmaceutical composition may further comprise one or more active agents. The pharmaceutical composition may further comprise one or more other therapeutic agents, such as lympho-depletive agents (e.g., thymoglobulin, campath-1H, anti-CD2 antibodies, anti-CD3 antibodies, anti-CD20 antibodies, cyclophosphamide, fludarabine), inhibitors of mTOR (e.g., sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18).

Depending upon the disease/condition and subject to be treated, as well as the route of administration, the LNP, cell, cell population or pharmaceutical composition may be administered at varying doses (e.g., measured in cells/kg or cells/subject). The physician in any event will determine the actual dosage which will be most suitable for any individual subject and it will vary with the age, weight and response of the particular subject. Typically, however, for the cells herein, doses of 5×10⁷ to 3×10⁹ cells, or 10⁸ to 2×10⁹ cells per subject may be administered.

The LNP, cell, cell population or pharmaceutical composition of the invention may be administered in one or more doses. It will be appreciated that due to the transient nature of the therapy, treatment of disease conditions requiring long term persistence of suppressive therapy may require the administration of more than one dose, e.g., at least 2, 3 or 4 doses, over a time period.

The cell or cell population may be appropriately modified for use in a pharmaceutical composition. For example, cells may be cryopreserved and thawed at an appropriate time, before being infused into a subject.

As described above, the LNP of the invention, or ex vivo transduced cells or cell populations can be used in vivo to provide a suppressive cell therapy to a subject in need thereof, e.g., a subject having an undesired inflammatory response, a subject suffering from an autoimmune condition, a subject with GvHD or HvG, or a subject in need of tissue remodeling or repair.

Thus, the invention provides a LNP, a CD4+ cell, a cell population or a pharmaceutical composition of the invention for use in therapy.

The LNPs, cells and compositions containing them are for adoptive cell therapy (ACT). Various conditions may be treated by administration of the LNPs, cells, and compositions of the invention. As noted above, these may be conditions responsive to immunosuppression, and particularly the immunosuppressive effects of Treg cells or of converted Treg-like cells. The LNPs, cells, cell populations, and compositions described herein may thus be used for inducing, or achieving, immunosuppression in a subject. The cells administered, or modified in vivo, may be targeted by expression of an antigen binding receptor, e.g., CAR. Conditions suitable for such treatment include infectious, neurodegenerative or inflammatory disease, or more broadly a condition associated with any undesired or unwanted or deleterious immune response. Further, the LNPs, cells, cell populations, and compositions described herein may thus be used to promote tissue remodeling or repair, e.g., to regenerate tissue.

Conditions to be treated or prevented include inflammation, or alternatively put, a condition associated with or involving inflammation. Inflammation may be chronic or acute. Furthermore, the inflammation may be low-level or systemic inflammation. For example, the inflammation may be inflammation which occurs in the context of a metabolic disorder, for example metabolic syndrome, or in the context of insulin resistance, or type II diabetes or obesity and such like.

Particularly conditions to be treated include conditions which may require rapid, broad immune modulation, such as acute liver dysfunction, sepsis, lung infection, autoimmune flares (acute flares of chronic diseases), stroke and vascular disease.

In particular, the LNPs, cells, cell populations, and pharmaceutical compositions provide a means for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation. The cells, cell populations, and pharmaceutical compositions may be used in a method which comprises the step of administering a LNP, cell, cell populations, or a pharmaceutical composition as described herein to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

For example, the LNP, cell, cell population or pharmaceutical composition of the invention, may be administered to a subject with a disease in order to lessen, reduce, or improve at least one symptom of disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The LNP, cell, cell population or pharmaceutical composition of the invention may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells are not administered, or progression of the disease may be completely stopped.

In one embodiment, the subject is a transplant recipient undergoing immunosuppression therapy.

Suitably, the subject is a mammal. Suitably, the subject is a human.

The transplant may be selected from a liver, kidney, heart, lung, pancreas, intestine, stomach, bone marrow, vascularized composite tissue graft, and skin transplant.

Suitably, the transduced cells may express a CAR which comprises an antigen binding domain which is capable of specifically binding to an HLA antigen that is present in the graft (transplant) donor but not in the graft (transplant) recipient.

Suitably, the transplant is a liver transplant. In embodiments where the transplant is a liver transplant, the antigen may be an HLA antigen present in the transplanted liver but not in the patient, a liver-specific antigen such as NTCP, or an antigen whose expression is up-regulated during rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

As discussed above, in one representative and preferred embodiment the antigen is HLA-A2.

A method for treating a disease or condition relates to the therapeutic use of the LNP, cell, cell population or pharmaceutical composition herein. In this respect, the LNP, cell, cell population or pharmaceutical composition of the invention may be administered to a subject having an existing disease or condition in order to lessen, reduce or improve at least one symptom associated with the disease or condition and/or to slow down, reduce or block the progression of the disease.

Suitably, treating and/or preventing cellular and/or humoral transplant rejection may refer to administering an effective amount of the LNP, cell, cell population or pharmaceutical composition of the invention such that the amount of immunosuppressive drugs that a transplant recipient requires is reduced, or may enable the discontinuation of immunosuppressive drugs.

Preventing a disease or condition relates to the prophylactic use of the LNP, cell, cell population or pharmaceutical composition of the invention herein. In this respect, the LNP, cell, cell population or pharmaceutical composition of the invention may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition.

The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis; neurodegenerative disease, for example, Amyotrophic Lateral Sclerosis (ALS); Chronic inflammatory demyelinating polyneuropathy (CIPD) and juvenile onset diabetes.

More particularly, the invention further provides an LNP, a CD4+ cell, a cell population or a pharmaceutical composition of the invention for use in treating or preventing inflammation, an autoimmune condition or HvG/GvHD or for promoting tissue remodeling or repair in a subject.

In a further aspect, the invention provides use of a LNP, a CD4+ cell, a cell population or a pharmaceutical composition of the invention in the manufacture of a medicament for treating or preventing inflammation, an autoimmune condition or HvG/GvHD or for promoting tissue remodeling or repair in a subject.

Further, the invention provides a method of treating or preventing inflammation, an autoimmune condition or HvG/GvHD or of promoting tissue remodeling or repair in a subject, comprising the step of administering a LNP, a CD4+ T cell, a cell population or a pharmaceutical composition of the invention to the subject.

An additional step of administration of low dose IL2 or of an IL2 mutein that may preferably bind to CD25 (e.g., those described previously) may be carried out to the subject prior at administration of a LNP, or pharmaceutical composition comprising a LNP of the invention. Such administration may increase the percentage or number of Treg cells in the patient prior to administration of the LNP. Particularly, the invention may provide a method of treating or preventing inflammation, an autoimmune condition or HvG/GvHD in a subject comprising the steps of (i) administering low dose IL2 or a IL2 mutein with enhanced or increased selectivity to CD25 to the subject and (ii) administering a LNP of the invention or a pharmaceutical composition comprising a LNP of the invention as described herein.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto. All publications mentioned herein are incorporated herein by reference.

```
Sequence listing
VH anti-CD4 antibody
SEQ ID NO. 1
EVQLVESGGGLVQPGRSLRLSCAASGFTFSNYGMAWVRQAPGKGLEWVATISYDGSITYY
RDSVKGRFTISRDNSKNTLYLQMNSLRAEDTAVYYCAREEQYSSWYFDFWGQGTLVTVSS
VL anti-CD4 antibody
SEQ ID NO. 2
DIQLTQSPSSLSASVGDRVTITCRASQSVSISSHDLMQWYQQKPGKAPKLLIYDAFNLASGV
PSRFSGSGSGTDFTLTISSLQPEDFATYYCQQSKDDPYTFGQGTKLEIK
Heavy chain anti-CD25 antibody
SEQ ID NO. 3
QVQLVQSGAEVKKPGSSVKVSCKASGYTFTSYRMHWVRQAPGQGLEWIGYINPSTGYTE
YNQKFKDKATITADESTNTAYMELSSLRSEDTAVYYCARGGGVFDYWGQGTLVTVSS
Light chain anti-CD25 antibody
SEQ ID NO. 4
DIQMTQSPSTLSASVGDRVTITCSASSSISYMHWYQQKPGKAPKLLIYTTSNLASGVPARFS
GSGSGTEFTLTISSLQPDDFATYYCHQRSTYPLTFGQGTKVEVK
Anti-CD4 aptamer
SEQ ID NO. 5
GGGAGACAAGAAT AAACGCTCAATGACGTCCTT AGAATTGCGCA
TTCCTCACACAGGATCTTTTCGACAGGAGGCTCACAACAGGC
FOXP3 amino acid sequence
SEQ ID NO. 6
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKRSQRPSR
CSNPTPGP
Variant FOXP3 amino acid sequence
SEQ ID NO. 7
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKREQRPSR
CSNPTPGP
Variant FOXP3 amino acid sequence
SEQ ID NO. 8
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKRSQRPAR
CSNPTPGP
Variant FOXP3 amino acid sequence
SEQ ID NO. 9
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKREQRPAR
CSNPTPGP
Truncated FOXP3 amino acid sequence
SEQ ID NO. 10
GGAHASSSSL NPMPPSQLQL PTLPLVMVAP SGARLGPLPH LQALLQDRPH
FMHQLSTVDA HARTPVLQVH PLESPAMISL TPPTTATGVF SLKARPGLPP GINVASLEWV
SREPALLCTF PNPSAPRKDS TLSAVPQSSY PLLANGVCKW PGCEKVFEEP
EDFLKHCQAD HLLDEKGRAQ CLLQREMVQS LEQQLVLEKE KLSAMQAHLA
GKMALTKASS VASSDKGSCC IVAAGSQGPV VPAWSGPREA PDSLFAVRRH
LWGSHGNSTF PEFLHNMDYF KFHNMRPPFT YATLIRWAIL EAPEKQRTLN
EIYHWFTRMF AFFRNHPATW KNAIRHNLSL HKCFVRVESE KGAVWTVDEL EF
FOXP3 polypeptide
SEQ ID NO. 11
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQVEELSAMQAHLAGKMALTKASSVASSDKGSCCIVAAGSQGPVVPAWSGPREAPDSLF
AVRRHLWGSHGNSTFPEFLHNMDYFKFHNMRPPFTYATLIRWAILEAPEKQRTLNEIYHWF
TRMFAFFRNHPATWKNAIRHNLSLHKCFVRVESEKGAVWTVDELEFRKKRSQRPSRCSNP
TPGPEGRGSLLTCGDVEEN
FOXP3 mRNA
SEQ ID NO. 12
AUG CCC AAC CCC AGG CCU GGC AAG CCC UCG GCC CCU UCC UUG GCC CUU
GGC CCA UCC CCA GGA GCC UCG CCC AGC UGG AGG GCU GCA CCC AAA GCC
UCA GAC CUG CUG GGG GCC CGG GGC CCA GGG GGA ACC UUC CAG GGC CGA
GAU CUU CGA GGC GGG GCC CAU GCC UCC UCU UCU UCC UUG AAC CCC AUG
CCA CCA UCG CAG CUG CAG CUG CCC ACA CUG CCC CUA GUC AUG GUG GCA
CCC UCC GGG GCA CGG CUG GGC CCC UUG CCC CAC UUA CAG GCA CUC CUC
CAG GAC AGG CCA CAU UUC AUG CAC CAG CUC UCA ACG GUG GAU GCC CAC
GCC CGG ACC CCU GUG CUG CAG GUG CAC CCC CUG GAG AGC CCA GCC AUG
AUC AGC CUC ACA CCA CCC ACC ACC GCC ACU GGG GUC UUC UCC CUC AAG
GCC CGG CCU GGC CUC CCA CCU GGG AUC AAC GUG GCC AGC CUG GAA UGG
GUG UCC AGG GAG CCG GCA CUG CUC UGC ACC UUC CCA AAU CCC AGU GCA
CCC AGG AAG GAC AGC ACC CUU UCG GCU GUG CCC CAG AGC UCC UAC CCA
CUG CUG GCA AAU GGU GUC UGC AAG UGG CCC GGA UGU GAG AAG GUC UUC
GAA GAG CCA GAG GAC UUC CUC AAG CAC UGC CAG GCG GAC CAU CUU CUG
GAU GAG AAG GGC AGG GCA CAA UGU CUC CUC CAG AGA GAG AUG GUA CAG
UCU CUG GAG CAG CAG CUG GUG CUG GAG AAG GAG AAG CUG AGU GCC AUG
CAG GCC CAC CUG GCU GGG AAA AUG GCA CUG ACC AAG GCU UCA UCU GUG
GCA UCA UCC GAC AAG GGC UCC UGC UGC AUC GUA GCU GCU GGC AGC CAA
GGC CCU GUC GUC CCA GCC UGG UCU GGC CCC CGG GAG GCC CCU GAC AGC
CUG UUU GCU GUC CGG AGG CAC CUG UGG GGU AGC CAU GGA AAC AGC ACA
UUC CCA GAG UUC CUC CAC AAC AUG GAC UAC UUC AAG UUC CAC AAC AUG CGA
CCC CCU UUC ACC UAC GCC ACG CUC AUC CGC UGG GCC AUC CUG GAG GCU
CCA GAG AAG CAG CGG ACA CUC AAU GAG AUC UAC CAC UGG UUC ACA CGC
AUG UUU GCC UUC UUC AGA AAC CAU CCU GCC ACC UGG AAG AAC GCC AUC
CGC CAC AAC CUG AGU CUG CAC AAG UGC UUU GUG CGG GUG GAG AGC GAG
AAG GGG GCU GUG UGG ACC GUG GAU GAG CUG GAG UUC CGC AAG AAA CGG
AGC CAG AGG CCC AGC AGG UGU UCC AAC CCU ACA CCU GGC CCC UGA
FOXP3 mRNA
SEQ ID NO. 13
GAAUUCGUCGACAUGCCCAACCCCAGACCCGGCAAGCCUUCUGCCCCUUCUCUGGC
CCUGGGACCAUCUCCUGGCGCCUCCCCAUCUUGGAGAGCCGCCCCUAAAGCCAGCG
AUCUGCUGGGAGCUAGAGGCCCUGGCGGCACAUUCCAGGGCAGAGAUCUGAGAGGC
GGAGCCCACGCCUCUAGCAGCAGCCUGAAUCCCAUGCCCCCUAGCCAGCUGCAGCU
GCCUACACUGCCUCUCGUGAUGGUGGCCCCUAGCGGAGCUAGACUGGGCCCUCUGC
CUCAUCUGCAGGCUCUGCUGCAGGACCGGCCCCACUUUAUGCACCAGCUGAGCACC
GUGGACGCCCACGCCAGAACACCUGUGCUGCAGGUGCACCCCCUGGAAAGCCCUGC
CAUGAUCAGCCUGACCCCUCCAACCACAGCCACCGGCGUGUUCAGCCUGAAGGCCA
GACCUGGACUGCCCCCUGGCAUCAAUGUGGCCAGCCUGGAAUGGGUGUCCCGCGAA
CCUGCCCUGCUGUGCACCUUCCCCAAUCCUAGCGCCCCCAGAAAGGACAGCACACU
GUCUGCCGUGCCCCAGAGCAGCUAUCCCCUGCUGGCUAACGGCGUGUGCAAGUGGC
CUGGCUGCGAGAAGGUGUUCGAGGAACCCGAGGACUUCCUGAAGCACUGCCAGGCC
GACCAUCUGCUGGACGAGAAAGGCAGAGCCCAGUGCCUGCUGCAGCGCGAGAUGGU
GCAGUCCCUGGAACAGCAGCUGGUGCUGGAAAAAGAAAAGCUGAGCGCCAUGCAGG
CCCACCUGGCCGGAAAGAUGGCCCUGACAAAAGCCAGCAGCGUGGCCAGCUCCGAC
AAGGGCAGCUGUUGUAUCGUGGCCGCUGGCAGCCAGGGACCUGUGGUGCCUGCUU
GGAGCGGACCUAGAGAGGCCCCCGAUAGCCUGUUUGCCGUGCGGAGACACCUGUG
GGGCAGCCACGGCAACUCUACCUUCCCCGAGUUCCUGCACAACAUGGACUACUUCAA
GUUCCACAACAUGAGGCCCCCCUUCACCUACGCCACCCUGAUCAGAUGGGCCAUUC
UGGAAGCCCCCGAGAAGCAGCGGACCCUGAACGAGAUCUACCACUGGUUUACCCGG
AUGUUCGCCUUCUUCCGGAACCACCCCGCCACCUGGAAGAACGCCAUCCGGCACAA
UCUGAGCCUGCACAAGUGCUUCGUGCGGGUGGAAAGCGAGAAGGGCGCCGUGUGG
ACAGUGGACGAGCUGGAAUUUCGGAAGAAGCGGUCCCAGAGGCCCAGCCGGUGUAG
CAAUCCUACACCUGGCCCUGAGGGCAGAGGAAGUCUGCUAACAUGCGGUGACGUCG
AGGAGAAUCC
anti-HLA A2 scFv
SEQ ID NO. 14
QVQLVQSGGGVVQPGGSLRVSCAASGVTLSDYGMHWVRQAPGKGLEWVAFIRNDGSDKYYADSVKGRFTISRD
NSEKTVSLQMSSLRAEDTAVYYCAKNGESGPLDYWYLDLWGRGTLVTVSSGGGGSGGGGSGGGGSTDVVMTQ
SPSSLSASVGDRVTITCQSSLDISHYLNWYQQKPGKAPKLLIYDASNLETGVPSRFSGSGSGTHFTFTISSLQ
PEDFATYYCQQYDNLPLTFGGGTKLEIK
CD8 hinge
SEQ ID NO. 15
TTTPAPRPPTPAPTIASQPLSLRPEACRPAAGGAVHTRGLDFACD
CD28 hinge
SEQ ID NO. 16
IEVMYPPPYLDNEKSNGTIIHVKGKHLCPSPLFPGPSKP
CD8 Transmembrane
SEQ ID NO. 17
IYIWAPLAGTCGVLLLSLVIT
CD28 Transmembrane
SEQ ID NO. 18
FWVLVVVGGVLACYSLLVTVAFIIFWV
CD3zeta intracellular signalling domain
SEQ ID NO. 19
RVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
CD28 costimulatory sequence
SEQ ID NO. 20
RSKRSRLLHSDYMNMTPRRPGPTRKHYQPYAPPRDFAAYRS
CD8 leader sequence
SEQ ID NO. 21
MALPVTALLLPLALLLHAARP
Helios polypeptide
SEQ ID NO. 22
METEAIDGYI TCDNELSPER EHSNMAIDLT SSTPNGQHAS PSHMTSTNSV
                60 70 80 90 100
KLEMQSDEEC DRKPISREDE IRGHDEGSSL EEPLIESSEV ADNRKVQELQ
              110 120 130 140 150
GEGGIRLPNG KLKCDVCGMV CIGPNVLMVH KRSHTGERPF HCNQCGASFT
              160 170 180 190 200
QKGNLLRHIK LHSGEKPFKC PFCSYACRRR DALTGHLRTH SVGKPHKCNY
              210 220 230 240 250
CGRSYKQRSS LEEHKERCHN YLQNVSMEAA GQVMSHHVPP MEDCKEQEPI
              260 270 280 290 300
MDNNISLVPF ERPAVIEKLT GNMGKRKSST PQKFVGEKLM RFSYPDIHFD
              310 320 330 340 350
MNLTYEKEAE LMQSHMMDQA INNAITYLGA EALHPLMQHP PSTIAEVAPV
              360 370 380 390 400
ISSAYSQVYH PNRIERPISR ETADSHENNM DGPISLIRPK SRPQEREASP
              410 420 430 440 450
SNSCLDSTDS ESSHDDHQSY QGHPALNPKR KQSPAYMKED VKALDTTKAP
              460 470 480 490 500
KGSLKDIYKV FNGEGEQIRA FKCEHCRVLF LDHVMYTIHM GCHGYRDPLE
              510 520
CNICGYRSQD RYEFSSHIVR GEHTFH
IL10 polypeptide
SEQ ID NO. 23
MHSSALLCCL VLLTGVRASP GQGTQSENSC THEPGNLPNM LRDLRDAFSR
                60 70 80 90 100
VKTFFQMKDQ LDNLLLKESL LEDFKGYLGC QALSEMIQFY LEEVMPQAEN
              110 120 130 140 150
QDPDIKAHVN SLGENLKTLR LRLRRCHRFL PCENKSKAVE QVKNAFNKLQ
              160 170
EKGIYKAMSE FDIFINYIEA YMTMKIRN
IL35 polypeptide
SEQ ID NO. 24
MCPARSLLLV ATIVLIDHLS LARNLPVATP DPGMFPCLHH SQNLLRAVSN
                60 70 80 90 100
MLQKARQTLE FYPCTSEEID HEDITKDKTS TVEACLPLEL TKNESCINSR
              110 120 130 140 150
ETSFITNGSC LASRKTSFMM ALCLSSIYED LKMYQVEFKT MNAKLLMDPK
              160 170 180 190 200
RQIFLDQNML AVIDELMQAL NENSETVPQK SSLEEPDFYK TKIKLCILLH
              210
AFRIRAVTID RVMSYLNAS
TGFbeta polypeptide
SEQ ID NO. 25
MPPSGLRLLL LLLPLLWLLV LTPGRPAAGL STCKTIDMEL VKRKRIEAIR
                60 70 80 90 100
GQILSKIRLA SPPSQGEVPP GPLPEAVLAL YNSTRDRVAG ESAEPEPEPE
              110 120 130 140 150
ADYYAKEVTR VLMVETHNEI YDKFKQSTHS IYMFFNTSEL REAVPEPVLL
              160 170 180 190 200
SRAELRLLRL KLKVEQHVEL YQKYSNNSWR YLSNRLLAPS DSPEWLSFDV
              210 220 230 240 250
TGVVRQWLSR GGEIEGFRLS AHCSCDSRDN TLQVDINGFT TGRRGDLATI
              260 270 280 290 300
HGMNRPFLLL MATPLERAQH LQSSRHRRAL DINYCFSSTE KNCCVRQLYI
              310 320 330 340 350
DERKDLGWKW IHEPKGYHAN FCLGPCPYIW SLDTQYSKVL ALYNQHNPGA
              360 370 380 390
SAAPCCVPQA LEPLPIVYYV GRKPKVEQLS NMIVRSCKCS
Areg polypeptide
SEQ ID NO. 26
MRAPLLPPAP VVLSLLILGS GHYAAGLDLN DTYSGKREPF SGDHSADGFE
                60 70 80 90 100
VTSRSEMSSG SEISPVSEMP SSSEPSSGAD YDYSEEYDNE PQIPGYIVDD
              110 120 130 140 150
SVRVEQVVKP PQNKTESENT SDKPKRKKKG GKNGKNRRNR KKKNPCNAEF
              160 170 180 190 200
QNFCIHGECK YIEHLEAVTC KCQQEYFGER CGEKSMKTHS MIDSSLSKIA
              210 220 230 240 250
LAAIAAFMSA VILTAVAVIT VQISHWSHCQ VIAINDESVL FPVDHKIMDP
              260 270
FCYDGFKLSI VTFYAISVYK GARR
CCN3 polypeptide
SEQ ID NO. 27
MQSVQSTSFC LRKQCLCLTF LLLHLLGQVA ATQRCPPQCP GRCPATPPTC
                60 70 80 90 100
APGVRAVLDG CSCCIVCARQ RGESCSDLEP CDESSGLYCD RSADPSNQTG
              110 120 130 140 150
ICTAVEGDNC VEDGVIYRSG EKFQPSCKFQ CTCRDGQIGC VPRCQLDVLL
              160 170 180 190 200
PEPNCPAPRK VEVPGECCEK WICGPDEEDS LGGLTLAAYR PEATLGVEVS
              210 220 230 240 250
DSSVNCIEQT TEWTACSKSC GMGFSTRVIN RNRQCEMLKQ TRLCMVRPCE
              260 270 280 290 300
QEPEQPTDKK GKKCLRTKKS LKAIHLQFKN CTSLHTYKPR FCGVCSDGRC
              310 320 330 340 350
CTPHNTKTIQ AEFQCSPGQI VKKPVMVIGT CTCHTNCPKN NEAFLQELEL
KTTRGKM
CD73 polypeptide
SEQ ID NO. 28
MCPRAARAPA TILLALGAVL WPAAGAWELT ILHINDVHSR LEQTSEDSSK
                60 70 80 90 100
CVNASRCMGG VARLFTKVQQ IRRAEPNVLL LDAGDQYQGT IWFTVYKGAE
              110 120 130 140 150
VAHFMNALRY DAMALGNHEF DNGVEGLIEP LIKEAKFPIL SANIKAKGPL
              160 170 180 190 200
ASQISGLYLP YKVLPVGDEV VGIVGYTSKE TPFLSNPGTN LVFEDEITAL
              210 220 230 240 250
QPEVDKLKTL NVNKIIALGH SGFEMDKLIA QKVRGVDVVV GGHSNTFLYT
              260 270 280 290 300
GNPPSKEVPA GKYPFIVTSD DGRKVPVVQA YAFGKYLGYL KIEFDERGNV
              310 320 330 340 350
ISSHGNPILL NSSIPEDPSI KADINKWRIK LDNYSTQELG KTIVYLDGSS
              360 370 380 390 400
QSCRFRECNM GNLICDAMIN NNLRHADETF WNHVSMCILN GGGIRSPIDE
              410 420 430 440 450
RNNGTITWEN LAAVLPFGGT FDLVQLKGST LKKAFEHSVH RYGQSTGEFL
              460 470 480 490 500
QVGGIHVVYD LSRKPGDRVV KLDVLCTKCR VPSYDPLKMD EVYKVILPNE
              510 520 530 540 550
LANGGDGFQM IKDELLRHDS GDQDINVVST YISKMKVIYP AVEGRIKEST
              560 570
GSHCHGSFSL IFLSLWAVIF VLYQ
CD39 polypeptide
SEQ ID NO. 29
MEDTKESNVK TFCSKNILAI IGFSSIIAVI ALLAVGLTQN KALPENVKYG
                60 70 80 90 100
IVLDAGSSHT SLYIYKWPAE KENDTGVVHQ VEECRVKGPG ISKFVQKVNE
              110 120 130 140 150
IGIYLTDCME RAREVIPRSQ HQETPVYLGA TAGMRLLRME SEELADRVLD
              160 170 180 190 200
VVERSLSNYP FDFQGARIIT GQEEGAYGWI TINYLLGKFS QKTRWFSIVP
              210 220 230 240 250
YETNNQETFG ALDLGGASTQ VTFVPQNQTI ESPDNALQFR LYGKDYNVYT
              260 270 280 290 300
HSFLCYGKDQ ALWQKLAKDI QVASNEILRD PCFHPGYKKV VNVSDLYKTP
              310 320 330 340 350
CIKRFEMTLP FQQFEIQGIG NYQQCHQSIL ELFNTSYCPY SQCAFNGIFL
              360 370 380 390 400
PPLQGDFGAF SAFYFVMKEL NLTSEKVSQE KVTEMMKKFC AQPWEEIKTS
              410 420 430 440 450
YAGVKEKYLS EYCESGTYIL SLLLQGYHFT ADSWEHIHFI GKIQGSDAGW
              460 470 480 490 500
TIGYMINLIN MIPAEQPLST PLSHSTYVFL MVLFSLVIFT VAIIGLLIFH
              510
KPSYFWKDMV
P2A peptide - cleavage domain
SEQ ID NO. 30
ATNFSLLKQAGDVEENPGP
T2A peptide - cleavage domain:
SEQ ID NO. 31
EGRGSLLTCGDVEENPGP
E2A peptide - cleavage domain:
SEQ ID NO. 32
QCTNYALLKLAGDVESNPGP
F2A peptide - cleavage domain:
SEQ ID NO. 33
VKQTLNFDLLKLAGDVESNPGP
Furin cleavage site
SEQ ID NO: 34
RXXR
Furin cleavage site
SEQ ID NO: 35
RRKR
CD4
SEQ ID NO: 36
MNRGVPFRHL LLVLQLALLP AATQGKKVVL GKKGDTVELT CTASQKKSIQ
                60 70 80 90 100
FHWKNSNQIK ILGNQGSELT KGPSKLNDRA DSRRSLWDQG NFPLIIKNLK
              110 120 130 140 150
IEDSDTYICE VEDQKEEVQL LVFGLTANSD THLLQGQSLT LTLESPPGSS
              160 170 180 190 200
PSVQCRSPRG KNIQGGKTLS VSQLELQDSG TWICTVLQNQ KKVEFKIDIV
              210 220 230 240 250
VLAFQKASSI VYKKEGEQVE FSFPLAFTVE KLTGSGELWW QAERASSSKS
              260 270 280 290 300
WITFDLKNKE VSVKRVIQDP KLQMGKKIPL HLTIPQALPQ YAGSGNITLA
              310 320 330 340 350
LEAKTGKLHQ EVNLVVMRAT QLQKNITCEV WGPTSPKLML SLKLENKEAK
              360 370 380 390 400
VSKREKAVWV LNPEAGMWQC LLSDSGQVLL ESNIKVLPTW STPVQPMALI
              410 420 430 440 450
VLGGVAGLLL FIGLGIFFCV RCRHRRRQAE RMSQIKRLLS EKKTCQCPHR
FQKTCSPI
pro IL2
SEQ ID NO. 37
MYRMQLLSCI ALSLALVINS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTEMCEYADE TATIVEFINR WITFCQSIIS
TLT
IL2
SEQ ID NO. 38
APTSSSTKKT QLQLEHLLLD LQMILNGINN
YKNPKLTRML TEKFYMPKKA TELKHLQCLE EELKPLEEVL NLAQSKNFHL
RPRDLISNIN VIVLELKGSE TTEMCEYADE TATIVEFLNR WITFCQSIIS
TLT
CDR3β sequence for GAD65(555-567): HLA-DR*04:01
SEQ ID NO. 39
CASSLVGGPSSEAFF
IL33
SEQ ID NO. 40
MKPKMKYSTN KISTAKWKNT ASKALCEKLG KSQQKAKEVC PMYFMKLRSG
                60 70 80 90 100
LMIKKEACYF RRETTKRPSL KTGRKHKRHL VLAACQQQST VECFAFGISG
              110 120 130 140 150
VQKYTRALHD SSITGISPIT EYLASLSTYN DQSITFALED ESYEIYVEDL
              160 170 180 190 200
KKDEKKDKVL LSYYESQHPS NESGDGVDGK MLMVTISPTK DEWLHANNKE
              210 220 230 240 250
HSVELHKCEK PLPDQAFFVL HNMHSNCVSF ECKTDPGVFI GVKDNHLALI
              260 270
KVDSSENLCT ENILFKLSET
CCN2
SEQ ID NO. 41
MTAASMGPVR VAFVVLLALC SRPAVGQNCS GPCRCPDEPA PRCPAGVSLV
                60 70 80 90 100
LDGCGCCRVC AKQLGELCTE RDPCDPHKGL FCHFGSPANR KIGVCTAKDG
              110 120 130 140 150
APCIFGGTVY RSGESFQSSC KYQCTCLDGA VGCMPICSMD VRLPSPDCPF
              160 170 180 190 200
PRRVKLPGKC CEEWVCDEPK DQTVVGPALA AYRLEDTFGP DPTMIRANCL
              210 220 230 240 250
VQTTEWSACS KTCGMGISTR VINDNASCRL EKQSRLCMVR PCEADLEENI
              260 270 280 290 300
KKGKKCIRTP KISKPIKFEL SGCTSMKTYR AKFCGVCTDG RCCTPHRTTT
              310 320 330 340
LPVEFKCPDG EVMKKNMMFI KTCACHYNCP GDNDIFESLY YRKMYGDMA
CTLA 4
SEQ ID NO. 42
MACLGFQRHK AQLNLAARTW PCTILFFLLF IPVECKAMHV AQPAVVLASS
                60 70 80 90 100
RGIASFVCEY ASPGKATEVR VTVLRQADSQ VTEVCAATYM MGNELTFLDD
              110 120 130 140 150
SICTGTSSGN QVNLTIQGLR AMDIGLYICK VELMYPPPYY LGIGNGTQIY
              160 170 180 190 200
VIDPEPCPDS DELLWILAAV SSGLFFYSFL LTAVSLSKML KKRSPLITGV
              210 220
YVKMPPTEPE CEKQFQPYFI PIN
LAG3
SEQ ID NO. 43
MWEAQFLGLL FLQPLWVAPV KPLQPGAEVP VVWAQEGAPA QLPCSPTIPL
                60 70 80 90 100
QDISLLRRAG VTWQHQPDSG PPAAAPGHPL APGPHPAAPS SWGPRPRRYT
              110 120 130 140 150
VLSVGPGGIR SGRLPLQPRV QLDERGRQRG DESLWLRPAR RADAGEYRAA
              160 170 180 190 200
VHLRDRALSC RLRLRLGQAS MTASPPGSLR ASDWVILNCS FSRPDRPASV
              210 220 230 240 250
HWFRNRGQGR VPVRESPHHH LAESFLFLPQ VSPMDSGPWG CILTYRDGEN
              260 270 280 290 300
VSIMYNLTVL GLEPPTPLTV YAGAGSRVGL PCRLPAGVGT RSFLTAKWTP
              310 320 330 340 350
PGGGPDLLVT GDNGDFTLRL EDVSQAQAGT YTCHIHLQEQ QLNATVTLAI
              360 370 380 390 400
ITVTPKSFGS PGSLGKLLCE VTPVSGQERF VWSSLDTPSQ RSFSGPWLEA
              410 420 430 440 450
QEAQLLSQPW QCQLYQGERL LGAAVYFTEL SSPGAQRSGR APGALPAGHI
              460 470 480 490 500
LIFLILGVIS LLLLVTGAFG FHLWRRQWRP RRFSALEQGI HPPQAQSKIE
              510 520
ELEQEPEPEP EPEPEPEPEP EPEQL
PDL1
SEQ ID NO. 44
                10 20 30 40 50
MRIFAVFIFM TYWHLLNAFT VTVPKDLYVV EYGSNMTIEC KFPVEKQLDL
                60 70 80 90 100
AALIVYWEME DKNIIQFVHG EEDLKVQHSS YRQRARLLKD QLSIGNAALQ
              110 120 130 140 150
ITDVKLQDAG VYRCMISYGG ADYKRITVKV NAPYNKINQR ILVVDPVTSE
              160 170 180 190 200
HELTCQAEGY PKAEVIWTSS DHQVLSGKTT TINSKREEKL ENVTSTIRIN
PDL2
SEQ ID NO. 45
                10 20 30 40 50
LQLHQIAALF TVTVPKELYI IEHGSNVTLE CNEDTGSHVN LGAITASLQK
                60 70 80 90 100
VENDTSPHRE RATLLEEQLP IGKASFHIPQ VQVRDEGQYQ CIIIYGVAWD
              110 120 130 140 150
YKYLTLKVKA SYRKINTHIL KVPETDEVEL TCQATGYPLA EVSWPNVSVP
              160 170 180 190 200
ANTSHSRTPE GLYQVTSVLR LKPPPGRNES CVFWNTHVRE LTLASIDLQS
              210 220 230 240 250
QMEPRTHPTW LLHIFIPSCI IAFIFIATVI ALRKQLCQKL YSSKDTTKRP
              260
VITTKREVNS AI
ICOS
SEQ ID NO. 46
                10 20 30 40 50
MKSGLWYFFL FCLRIKVLIG EINGSANYEM FIFHNGGVQI LCKYPDIVQQ
                60 70 80 90 100
FKMQLLKGGQ ILCDLTKTKG SGNTVSIKSL KFCHSQLSNN SVSFFLYNLD
              110 120 130 140 150
HSHANYYFCN LSIFDPPPEK VTLIGGYLHI YESQLCCQLK FWLPIGCAAF
              160 170 180 190
VVVCILGCIL ICWLTKKKYS SSVHDPNGEY MFMRAVNTAK KSRLTDVTL
Heavy chain of anti-CD62L antibody
SEQ ID NO. 47
QVQLVQSGAEVKKPGSSVKVSCKASGYTFTSYVMHWVRQAPGQGLEWIGYIYPYNDGTK
YNEKFKGRVTITSDESTNTAYMELSSLRSEDTAVYYCAREEYGNYVRYFDVWGQGTLVTV
SS
Light chain of anti-CD62L antibody
SEQ ID NO. 48
DIQMTQSPSTLSASVGDRVTITCKSSQSLLNSSNQKNYLAWYQQKPGKAPKLLVYFASTRE
SGVPDRFIGSGSGTDFTLTISSLQPEDFATYFCHQHYSTPLTFGQGTKVEVK
IL37
SEQ ID NO. 49
VHTSP
                60 70 80 90 100
KVKNLNPKKF SIHDQDHKVL VIDSGNLIAV PDKNYIRPEI FFALASSLSS
              110 120 130 140 150
ASAEKGSPIL LGVSKGEFCL YCDKDKGQSH PSLQLKKEKL MKLAAQKESA
              160 170 180 190 200
RRPFIFYRAQ VGSWNMLESA AHPGWFICTS CNCNEPVGVT DKFENRKHIE
              210
FSFQPVCKAE MSPSEVSD
IL22
SEQ ID NO. 50
APISSHC RLDKSNFQQP
                60 70 80 90 100
YITNRTEMLA KEASLADNNT DVRLIGEKLE HGVSMSERCY LMKQVINFTL
              110 120 130 140 150
EEVLFPQSDR FQPYMQEVVP FLARLSNRLS TCHIEGDDLH IQRNVQKLKD
              160 170
TVKKLGESGE IKAIGELDLL FMSLRNACI
Heavy chain of anti-CD62L antibody (alternative)
SEQ ID NO. 51
EVQLQQSGPDLVKPGASVKMSCKASGYTFTSYVMHWVKQKPGQGLEWIGYIYPYNDGTKYNEKFKGKATLTSDK
SSSTAYMELSSLTSEDSAVYYCAREEYGNYVRYFDVWGAGTTVTVSS
Light chain of anti-CD62L antibody (alternative)
SEQ ID NO. 52
DIVMTQSPSSLAMSVGQKVTMTCKSSQSLLNSSNQKNYLAWYQQKPGQSPKLLVYFASTRESGVPDRFIGSGSGT
DFTLTISSVQAEDLADYFCHQHYSTPLTFGAGTKLELK
```

### EXAMPLES

### Example 1

mRNA will be manufactured by in vitro transcription using a T7 promoter containing plasmid with a 120 bp polyA tail encoding FOXP3 of SEQ ID NO. 6 and a chimeric antigen receptor comprising an scFv of SEQ ID NO. 14. The plasmid will be linearized and transcribed in vitro using the mMESSAGE mMACHINE T7 ULTRA kit (Life technologies), incorporating 10% 2-Thio-UTP and 5-Methyl-CTP (TriLink Bio Technologies).

Capping of the mRNA will be carried out by using the trinucleotide cap1 analog Clean Cap (TriLink). mRNA will be purified by cellulose purification and stored at −20° C.

Purified mRNA will be encapsulated in LNP by using an ethanolic lipid mixture of a cationic lipid, phosphatidylcholine, cholesterol and polyethylene glycol-lipid. The mixture will be rapidly mixed with an aqueous solution of the mRNA at acidic pH and particles will be characterized using a Zetasizer Nano ZS (Malvern Instruments).

To prepare CD4 targeting LNPs, the LNPs will be conjugated to an anti-CD4 antibody using SATA-maleimide chemistry. LNP will be modified with maleimide functioning groups and the antibody will be functionalized with SATA to introduce sulfhydryl groups. Thioester conjugation chemistry will be used to bind the antibody to the LNP. Purification can then be carried out using Sepharose CL-4B gel filtration columns. The LNPs will be kept at 4° C.

### Example 2

The LNPs will then be tested in vitro to assess cell specificity and expression efficacy. CD4-targeted LNPs containing modified mRNA encoding an HLA.A2-CAR and FOXP3 will be incubated with freshly isolated human PBMCs. The cell culture will be activated with anti-CD3/CD28 beads to promote T cell activation. Phenotypic and functional characterization of the cells will be performed 24, 48 and 72 hours after exposure to the CD4/LNP-HLAA2-FOXP3. LNP conjugated with an isotype control antibody will be used to assess the unspecific delivery of mRNA into non-CD4 cell types.

Protein expression of HLA.A2-CAR and FOXP3 will be assessed by Flow Cytometry at multiple time points to evaluate targeting specificity and expression efficacy. In addition, multiple markers associated to regulatory T cell (Treg) function will be analyzed to determine the phenotypic changes of the targeted cells (ie. CTLA4, HELIOS, CD25, CD127, PD1). Functional characterization of cell exposed to CD4/LNP-HLAA2-FOXP3 will be performed on isolated HLA.A2-CAR expressing cells. Antigen-specific activation will be tested by culturing the isolated cells with K562 cells expressing or not HLA.A2 molecules and assessing CD69 and CD137 activation markers at 24 hours. Cell viability of the K562 target cell will be assessed to evaluate and exclude cytotoxicity of the transformed cells. In addition, culture supernatants will be collected to investigate the content of anti- and pro-inflammatory cytokines.

The suppressive function of the transformed cells will be further assessed in vitro in a co-culture with HLA-A2 expressing B cells and CD4+CD25−effector T cells. Briefly, CD4+CD25− T cells will be isolated and cultured with a B cells line expressing HLA-A2 molecules. Transformed CD4/LNP-HLAA2-FOXP3 cells will add to the culture at different cell:cell ratios, and proliferation of the CD4+CD25− T cells will be assessed 5 days later.

### Example 3

To assess the capacity of the CD4/LNP-HLAA2-FOXP3 to efficiently reprogram human T cells in vivo, PBMCs from an HLA.A2 negative donor will be transferred to NSG.HLA.A2 mice and CD4/LNP-HLAA2-FOXP3 particles will be injected 2 weeks later. Phenotypic characterization of cells from blood and spleen will be performed 24, 48 and 96 hours after intravenous injection of the LNPs. Expression levels of HLA.A2 CAR and FOXP3 on CD4+ T cells and other cells will be assessed to determine specificity, efficacy and expression persistence.

In addition, this same model will be used to assess the immune modulation capacity of the CD4/LNP-HLAA2-FOXP3 to control the development of xenoGvHD. Three groups of NSG.HLA.A2 mice will be transferred with human PBMCs and followed for signs of GvHD for 8 weeks. The first group will only receive PBMCs, while the other two groups will receive respectively 1 or 2 doses of intravenous injection of CD4/LNP-HLAA2-FOXP3 (1 dose at week-2, or 1 dose at week-2+1 dose at week-3). Scoring will be performed daily in all animals to assess the development of xenoGvHD. In addition, tail-blood will be isolated every week to track the engraftment of human cells (ie. frequency of human-CD45 vs mouse-CD45) and assess the reprograming of human T cells in circulation (ie. frequency of CD4+HLA.A2+FOXP3+).

1. A lipid nanoparticle comprising mRNA encoding FOXP3 and at least a second polypeptide, wherein said lipid nanoparticle comprises a moiety capable of specifically binding to a molecule expressed on a CD4+ T cell and wherein said mRNA is modified to have increased stability as compared to unmodified mRNA.

2. The lipid nanoparticle of claim 1 wherein the moiety is capable of binding to CD4, CD25, CD62L or CD45RA.

3. The lipid nanoparticle of claim 1 or 2, wherein the LNP specifically binds to a CD4+ T cell.

4. The lipid nanoparticle of claim 3, wherein the CD4+ T cell is a Treg.

5. The lipid nanoparticle of anyone of claims 1 to 4, wherein the moiety is an antibody, antibody fragment, or aptamer.

6. The lipid nanoparticle of anyone of claims 1 to 5 wherein the mRNA encodes at least 3, 4 or 5 polypeptides.

7. The lipid nanoparticle of anyone of claims 1 to 6 wherein the at least a second polypeptide is selected from any one or more of an antigen binding receptor, a transcription factor, a persistence associated polypeptide, a cytokine, a safety switch polypeptide or a polypeptide which promotes tissue repair or suppressive function.

8. The lipid nanoparticle of claim 7 wherein the antigen binding receptor is a CAR or TCR.

9. The lipid nanoparticle of claim 7 or 8 wherein the antigen binding receptor binds to HLA-A2.

10. The lipid nanoparticle of any one of claims 7 to 9 wherein the antigen binding receptor comprises SEQ ID NO. 14 or a functional variant thereof.

11. The lipid nanoparticle of claim 7 wherein the transcription factor is selected from the group consisting of Helios, Sp1, NFAT, AP1, STAT5, CREB, ATF or Smad.

12. The lipid nanoparticle of claim 7, wherein the persistence associated polypeptide is a STAT5 or JAK1 polypeptide comprising a gain of function mutation or comprises a STAT5 association motif and a JAK1 and/or JAK2 binding motif.

13. The lipid nanoparticle of claim 7 wherein the cytokine is IL10, IL35 or TGFβ.

14. The lipid nanoparticle of claim 7, wherein the safety switch polypeptide comprises a CD20 epitope.

15. The lipid nanoparticle of claim 7 wherein the polypeptide which promotes tissue repair is amphiregulin or CCN3.

16. The lipid nanoparticle of claim 7 wherein the polypeptide which promotes suppressive function is CD73 or CD39.

17. The lipid nanoparticle of any one of claims 1 to 16 wherein the mRNA comprises a 5′ cap and a polyA tail.

18. The lipid nanoparticle of any one of claims 1 to 17 wherein the mRNA comprises one or more modified nucleotides.

19. The lipid nanoparticle of any one of claims 1 to 18 wherein the lipid nanoparticle is formulated from an ionizable or cationic lipid and optionally comprises DOPE and/or cholesterol.

20. A composition comprising a CD4+ T cell and a LNP of any one of claims 1 to 19.

21. A CD4+ T cell comprising a LNP of any one of claims 1 to 19.

22. An ex vivo method of transducing a CD4+ T cell comprising the step of combining a CD4+ T cell with a LNP of any one of claims 1 to 19.

23. A transduced CD4+ T cell obtainable or obtained by the method of claim 22.

24. The cell of claim 21 or 23 wherein the cell is a T reg.

25. A cell population comprising a cell of claim 21, 23 or 24.

26. A pharmaceutical composition comprising a LNP of any one of claims 1 to 19, a cell of claim 21, 23 or 24, or a cell population of claim 25 comprising one or more pharmaceutically acceptable excipients.

27. A LNP of any one of claims 1 to 19, a cell of claim 21, 23 or 24, a cell population of claim 25 or a pharmaceutical composition of claim 26 for use in therapy

28. A LNP of any one of claims 1 to 19, a cell of claim 21, 23 or 24, a cell population of claim 25 or a pharmaceutical composition of claim 26 for use in treating or preventing inflammation, an autoimmune condition or HvG/GvHD in a subject.

29. Use of a LNP of any one of claims 1 to 19, a cell of claim 21, 23 or 24, a cell population of claim 25 or a pharmaceutical composition of claim 26 in the manufacture of a medicament for treating or preventing inflammation, an autoimmune condition or HvG/GVHD in a subject.

30. A method of producing a LNP of any one of claims 1 to 19 comprising the step of incubating an ionizable or cationic lipid with mRNA encoding FOXP3 and a second polypeptide wherein said mRNA is modified to have increased intracellular stability as compared to unmodified mRNA, to create a LNP-mRNA formulation.


SEQUENCE LISTING (52 sequences)

SEQ ID NO: 1 - protein, 120 aa, synthetic construct
  source 1..120: note = VH anti-CD4 antibody
EVQLVESGGGLVQPGRSLRLSCAASGFTFSNYGMAWVRQAPGKGLEWVATISYDGSITYYRDSVKGRFTISRDNSKNTLYLQMNSLRAEDTAVYYCAREEQYSSWYFDFWGQGTLVTVSS

SEQ ID NO: 2 - protein, 111 aa, synthetic construct
  source 1..111: note = VL anti-CD4 antibody
DIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIK

SEQ ID NO: 3 - protein, 116 aa, synthetic construct
  source 1..116: note = Heavy chain anti-CD25 antibody
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 4 - protein, 106 aa, synthetic construct
  source 1..106: note = Light chain anti-CD25 antibody
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 5 - protein, 86 aa, synthetic construct
  source 1..86: note = Anti-CD4 aptamer
GGGAGACAAGAATAAACGCTCAATGACGTCCTTAGAATTGCGCATTCCTCACACAGGATCTTTTCGACAGGAGGCTCACAACAGGC

SEQ ID NO: 6 - protein, 431 aa, Homo sapiens
  source 1..431: note = FOXP3 amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 7 - protein, 431 aa, synthetic construct
  source 1..431: note = Variant FOXP3 amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPSRCSNPTPGP

SEQ ID NO: 8 - protein, 431 aa, synthetic construct
  source 1..431: note = Variant FOXP3 amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPARCSNPTPGP

SEQ ID NO: 9 - protein, 431 aa, synthetic construct
  source 1..431: note = Variant FOXP3 amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPARCSNPTPGP

SEQ ID NO: 10 - protein, 362 aa, synthetic construct
  source 1..362: note = Truncated FOXP3 amino acid sequence
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 11 - protein, 441 aa, synthetic construct
  source 1..441: note = FOXP3 polypeptide
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 12 - RNA, 1296 nt, Homo sapiens
  source 1..1296: mol_type = mRNA; note = FOXP3 mRNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 13 - RNA, 1352 nt, Homo sapiens
  source 1..1352: mol_type = mRNA; note = FOXP3 mRNA
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 14 - protein, 246 aa, synthetic construct
  source 1..246: note = anti-HLA A2 scFv
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 15 - protein, 45 aa, synthetic construct
  source 1..45: note = CD8 hinge
TTTPAPRPPTPAPTIASQPLSLRPEACRPAAGGAVHTRGLDFACD

SEQ ID NO: 16 - protein, 39 aa, synthetic construct
  source 1..39: note = CD28 hinge
IEVMYPPPYLDNEKSNGTIIHVKGKHLCPSPLFPGPSKP

SEQ ID NO: 17 - protein, 21 aa, synthetic construct
  source 1..21: note = CD8 Transmembrane
IYIWAPLAGTCGVLLLSLVIT

SEQ ID NO: 18 - protein, 27 aa, synthetic construct
  source 1..27: note = CD28 Transmembrane
FWVLVVVGGVLACYSLLVTVAFIIFWV

SEQ ID NO: 19 - protein, 112 aa, synthetic construct
  source 1..112: note = CD3zeta intracellular signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 41 aa, synthetic construct
  source 1..41: note = CD28 costimulatory sequence
RSKRSRLLHSDYMNMTPRRPGPTRKHYQPYAPPRDFAAYRS

SEQ ID NO: 21 - protein, 21 aa, synthetic construct
  source 1..21: note = CD8 leader sequence
MALPVTALLLPLALLLHAARP

SEQ ID NO: 22 - protein, 526 aa, synthetic construct [sequence omitted]

SEQ ID NO: 23 - protein, 178 aa, synthetic construct
  source 1..178: note = IL10 polypeptide
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 24 - protein, 219 aa, synthetic construct
  source 1..219: note = IL35 polypeptide
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - protein, 390 aa, synthetic construct
  source 1..390: note = TGFbeta polypeptide
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - protein, 274 aa, synthetic construct
  source 1..274: note = Areg polypeptide
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQISHWSHCQVIAINDESVLFPVDHKTMDPFCYDGFKLSIVTFYAISVYKGARR

SEQ ID NO: 27 - protein, 357 aa, synthetic construct
  source 1..357: note = CCN3 polypeptide
MQSVQSTSFCLRKQCLCLTFLLLHLLGQVAATQRCPPQCPGRCPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 28 - protein, 574 aa, synthetic construct [sequence omitted]

SEQ ID NO: 29 - protein, 510 aa, synthetic construct
  source 1..510: note = CD39 polypeptide
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 30 - protein, 19 aa, synthetic construct
  source 1..19: note = P2A peptide - cleavage domain
ATNFSLLKQAGDVEENPGP

SEQ ID NO: 31 - protein, 18 aa, synthetic construct
  source 1..18: note = T2A peptide - cleavage domain
EGRGSLLTCGDVEENPGP

SEQ ID NO: 32 - protein, 20 aa, synthetic construct
  source 1..20: note = E2A peptide - cleavage domain
QCTNYALLKLAGDVESNPGP

SEQ ID NO: 33 - protein, 22 aa, synthetic construct
  source 1..22: note = F2A peptide - cleavage domain
VKQTLNFDLLKLAGDVESNPGP

SEQ ID NO: 34 - skipped

SEQ ID NO: 35 - protein, 4 aa, synthetic construct
  source 1..4: note = Furin cleavage site
RRKR

SEQ ID NO: 36 - protein, 458 aa, synthetic construct
  source 1..458: note = CD4
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 37 - protein, 153 aa, synthetic construct
  source 1..153: note = pro IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 38 - protein, 133 aa, synthetic construct [sequence omitted]

SEQ ID NO: 39 - protein, 15 aa, synthetic construct
  source 1..15: note = CDR3beta sequence for GAD65(555-567):HLA-DR*04:01
CASSLVGGPSSEAFF

SEQ ID NO: 40 - protein, 270 aa, synthetic construct
  source 1..270: note = IL33
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 41 - protein, 349 aa, synthetic construct
  source 1..349: note = CCN2
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 42 - protein, 223 aa, synthetic construct [sequence omitted]

SEQ ID NO: 43 - protein, 525 aa, synthetic construct
  source 1..525: note = LAG3
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 44 - protein, 290 aa, synthetic construct
  source 1..290: note = PDL1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 45 - protein, 262 aa, synthetic construct
  source 1..262: note = PDL2
LQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDTTKRPVTTTKREVNSAI

SEQ ID NO: 46 - protein, 199 aa, synthetic construct
  source 1..199: note = ICOS
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 47 - protein, 121 aa, synthetic construct
  source 1..121: note = Heavy chain of anti-CD62L antibody
QVQLVQSGAEVKKPGSSVKVSCKASGYTFTSYVMHWVRQAPGQGLEWIGYIYPYNDGTKYNEKFKGRVTITSDESTNTAYMELSSLRSEDTAVYYCAREEYGNYVRYFDVWGQGTLVTVSS

SEQ ID NO: 48 - protein, 113 aa, synthetic construct
  source 1..113: note = Light chain of anti-CD62L antibody
DIQMTQSPSTLSASVGDRVTITCKSSQSLLNSSNQKNYLAWYQQKPGKAPKLLVYFASTRESGVPDRFIGSGSGTDFTLTISSLQPEDFATYFCHQHYSTPLTFGQGTKVEVK

SEQ ID NO: 49 - protein, 173 aa, synthetic construct
  source 1..173: note = IL37
VHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 50 - protein, 146 aa, synthetic construct
  source 1..146: note = IL22
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 51 - protein, 121 aa, synthetic construct
  source 1..121: note = Heavy chain of anti-CD62L antibody (alternative)
EVQLQQSGPDLVKPGASVKMSCKASGYTFTSYVMHWVKQKPGQGLEWIGYIYPYNDGTKYNEKFKGKATLTSDKSSSTAYMELSSLTSEDSAVYYCAREEYGNYVRYFDVWGAGTTVTVSS

SEQ ID NO: 52 - protein, 113 aa, synthetic construct
  source 1..113: note = Light chain of anti-CD62L antibody (alternative)
DIVMTQSPSSLAMSVGQKVTMTCKSSQSLLNSSNQKNYLAWYQQKPGQSPKLLVYFASTRESGVPDRFIGSGSGTDFTLTISSVQAEDLADYFCHQHYSTPLTFGAGTKLELK